(19) United States
(12) Patent Application Publication
(10) Pub. No.: US 2022/0079795 A1
(43) Pub. Date: Mar. 17, 2022

(54) SPINAL ALIGNMENT COMPONENT

(71) Applicants: Maciej GROSS, London (GB); Toby ATKINSON, London (GB)

(72) Inventors: Maciej GROSS, London (GB); Toby ATKINSON, London (GB)

(21) Appl. No.: 17/612,874

(22) PCT Filed: May 18, 2020
(86) PCT No.: PCT/EP2020/063834

§ 371 Date: Nov. 19, 2021

(30) Foreign Application Priority Data
May 20, 2019 (GB) 1907095.2

(51) Int. Cl. A61F 5/02; A61B 5/00; A45F 3/04; A45F 3/00

(52) U.S. Cl. CPC A61F 5/026; A61B 5/6831; A61B 5/6812; A45F 2003/001; A45F 3/04; A61B 5/6843; A61F 5/028

(57) ABSTRACT

A spinal alignment component is adapted for incorporation into a wearable spinal alignment apparatus and comprises a first rigid elongate member comprising a first distal end and a second rigid elongate member comprising a second distal end. The first rigid elongate member and the second rigid elongate member are independently movable relative to one another to adjust the displacement between the first distal end and the second distal end. Wearable spinal alignment apparatus incorporating the component is also described. The component and apparatus offers a means to encourage ideal alignment and correct the posture of a wearer.

### RELATED APPLICATION

This application is related to and claims priority from United Kingdom patent application number 1907095.2 filed 20 May 2019, the contents of which are incorporated herein by reference in their entirety.

### TECHNICAL FIELD

The present invention relates to a spinal alignment component and, more particularly, to a spinal alignment component comprising first and second rigid elongate members which are independently movable. The invention also relates to wearable alignment devices, such as wearable spinal alignment apparatus, comprising the spinal alignment component and uses of the spinal alignment component for correcting posture during everyday activities and exercise.

### BACKGROUND OF THE INVENTION

Poor posture may lead to poor health, sickness, pain and injury. Workforce productivity is reduced due to lost work days and quality of life is compromised by avoidable injury.

Correct posture is brought about by ideal alignment. Ideal alignment is the position adopted when the apex of the sacral spine, the apex of the thoracic spine and the back of the occipital head are collinear. Ideal alignment arranges the body's major axis so that the major axis may be transected by a single vertical line.

Conventionally, training ideal alignment comprises instruction by a postural expert (e.g. a doctor or physiotherapist). A patient is instructed to stand with their back against a straight rigid object (such as a wall, a pole, or a broom handle) so the patient's head, shoulders and pelvis are in contact with the rigid object. Disadvantages of this approach include the requirement for a postural expert, the lack of mobility during training and that the method is not hands free. Therefore, training is restricted to short time periods and while not carrying out everyday activities or exercise.

Other approaches use a wearable device. These devices aim to secure a straight rigid object (corresponding to a wall or pole) to the back of the wearer at the head, shoulders and pelvis. The securement means are often disposed about the head, torso and abdomen, therefore restricting the wearer's rotation and flexion at the head and hips.

The securement means often comprise elastic or tensioning straps, which assist the movement of the wearer into ideal alignment. For example, a force is exerted on the head, shoulders and abdomen which is substantially horizontal and toward the rigid member, and a force is exerted on the cervical spine which is substantially horizontally and away from the rigid member. The force constrains the wearer to ideal alignment. As a result, the wearer does not adapt mentally or physically to maintain ideal alignment and will therefore become reliant on the device to provide ideal alignment.

Furthermore, known devices are cumbersome and difficult to attach to the wearer, necessitating assistance from a third party for a wearer wishing to wear the device. This is not always practical since a wearer may wish to use such a device on a daily basis for optimum long-term posture improvement results. Existing devices are not intended for personal or home use, but for use in the presence of e.g. a doctor or physiotherapist.

Such devices are often of a fixed size. They are large and not compactable, making storage difficult when not in use. Fixed size devices are only suitable for a narrow size range of wearers.

Other devices incorporate a means to communicate to the wearer when correct or incorrect posture is achieved. These devices often focus on the posture of one portion of the body, such as the abdomen, and do not result in ideal alignment of the whole body. During everyday activities and exercise, unaccounted for changes in position result in the device communicating false feedback. This confuses the wearer, renders the device unusable during such activity and may encourage non-ideal alignment and incorrect posture.

There is a need for a device which offers an improved means to encourage ideal alignment and correct posture in all aspects of life, while not interfering with movement and flexibility of the wearer during everyday activities and exercise, and also not making the wearer reliant on the device to help them achieve ideal alignment. There is also a need for versatile devices which can be easily put in place, adjusted in size, removed and stored by the wearer.

### SUMMARY OF THE INVENTION

A first aspect of the invention is a spinal alignment component adapted for incorporation into a wearable spinal alignment apparatus, the spinal alignment component comprising:

a first rigid elongate member comprising a first distal end; and

a second rigid elongate member comprising a second distal end; wherein the first rigid elongate member and the second rigid elongate member are independently movable relative to one another to adjust the displacement between the first distal end and the second distal end.

The spinal alignment component is a discrete component or unit which can be easily incorporated into a wide range of wearable apparatus, including but not limited to backpacks (also referred to as rucksacks or knapsacks), harnesses or clothing, such as coats or jackets. The invention therefore provides a versatile device usable in a variety of different settings to help improve posture.

The component is used as an aid to improve posture when incorporated into wearable spinal alignment apparatus and worn by a user. The first rigid elongate member comprises a first contact portion, the second elongate member comprises a second contact portion and the component further comprises a third contact portion. When the component is incorporated into a wearable spinal alignment apparatus which is worn by a wearer, the component is adjustable such that first, second and third contact portions are arranged collinearly and disposed to contact the wearer's occipital head, sacral spine and thoracic spine respectively. The three contact portions are collinear, so that when contact is achieved by the wearer's occipital head, sacral spine and thoracic spine with the contact portions, then the wearer is in ideal alignment. These contact portions are disposed along the length of the component, so that when contact is achieved the contact portions remain collinear and provide tactile feedback to the wearer. The rigidity (or stiffness) of the elongate members of the component ensures that, during use, the three contact portions remain substantially collinear, even when the component is subjected to the normal forces associated with being worn. In other words, the rigidity ensures that the wearer can maintain ideal alignment during use when contact with all three contact portions is felt. The wearer uses the tactile feedback to guide them to achieve simultaneous contact at all three points and therefore achieve ideal alignment.

A second aspect of the invention is a wearable spinal alignment apparatus comprising the spinal alignment component according to the first aspect, and a securement system to attach the apparatus to the torso of a wearer.

The wearable spinal alignment apparatus of the second aspect facilitates the wearing of the spinal alignment component so that the wearer can achieve the posture improvement benefits described above. The design of the wearable spinal alignment apparatus is not limited provided that is provides for the component to be securely held to the back of the wearer such that contact of one or more of the first, second and third contact portions with the wearer's occipital head, sacral spine and thoracic spine respectively is possible. Non-limiting examples of the wearable spinal alignment apparatus comprise the spinal alignment component incorporated into apparatus selected from a backpack, a harness or a piece of clothing (for example a shirt, blouse, t-shirt, coat, jacket or sweatshirt).

A third aspect of the invention is the use of the spinal alignment component according to the first aspect in a wearable spinal alignment apparatus.

A fourth aspect of the invention is the use of the spinal alignment component according to the first aspect in the manufacture of a wearable spinal alignment apparatus.

A fifth aspect of the invention is the use of the spinal alignment component according to the first aspect to improve or correct the posture of a user.

A sixth aspect of the invention is the use of the spinal alignment component according to the first aspect in a wearable alignment device or apparatus.

A seventh aspect of the invention is a wearable alignment device, comprising a rigid elongate member comprising first, second and third contact portions disposed along its length, such that when the device is worn by a wearer the first, second and third contact portions are arranged collinearly and disposed to contact the wearer's occipital head, sacral spine and thoracic spine respectively; and a securement system comprising left and right shoulder loops adapted to encircle the wearer's left and right shoulders, and at least one horizontal securement strap adapted to connect the left and right shoulder loops across the wearer's front; wherein when the device is worn by a wearer the securement system is disposed to secure the third contact portion in contact with the thoracic spine, and the first and second contact portions remain movable away from the occipital head and sacral spine respectively.

In the device according to the seventh aspect the three contact portions are collinear, so that when contact is achieved by the wearer's occipital head, sacral spine and thoracic spine with the contact portions, then the wearer is in ideal alignment. These contact portions are disposed along the length of a rigid elongate member, so that when contact is achieved the contact portions remain collinear and provide tactile feedback to the wearer. The rigidity (or stiffness) of the elongate member ensures that, during use, the three contact portions on the elongate member remain substantially collinear, even when the elongate member is subjected to the normal forces associated with being worn. In other words, the rigidity ensures that the wearer can maintain ideal alignment during use when contact with all three contact portions is felt. The wearer uses the tactile feedback to guide them to achieve simultaneous contact at all three points and therefore achieve ideal alignment.

The securement system only secures the third contact portion in contact with the thoracic spine, while the first and second contact portions are not secured in contact with the head and sacral spine respectively, but are held adjacent to and remain movable away from the head and sacral spine respectively.

As a result, the wearer's movement is minimally restricted by the securement system, the absence of securement straps encircling the wearer's waist and head allows greater flexion of the spine and neck, so everyday activities and exercise are not hindered.

Also, the absence of securement straps encircling the wearer's waist and head, means the wearer is not forced into ideal alignment, but must independently move their body into ideal alignment by making contact with the first, second and third contact portions of the device.

Independent movement to achieve ideal alignment (compared to forced movement) requires more repetitive and persistent mental and physical action by the wearer. A more repetitive and persistent action increases adaption, resulting in superior muscle memory. Advantageously, because the wearer is effectively mentally and physically adapting to maintain ideal alignment and correct posture, the present invention stops the wearer becoming reliant on an alignment or posture training device.

An eighth aspect of the invention is a method for using the wearable alignment device according to the seventh aspect to improve the alignment of a wearer.

A ninth aspect of the invention is a method of manufacturing the wearable alignment device according to the seventh aspect.

A tenth aspect of the invention is a bag comprising the wearable alignment device according to the seventh aspect. In some embodiments, the bag is a rucksack.

An eleventh aspect of the invention is clothing comprising the wearable alignment device according to the seventh aspect. In some embodiments, the clothing comprises a vest or gilet.

A twelfth aspect of the invention is a spinal alignment component adapted for incorporation into a wearable spinal alignment apparatus, wherein the spinal alignment component comprises sensing means to detect one or more of movement, position and orientation of a wearer during use of the component.

In this way a component is provided which is able to guide a user to adopt optimal body position (e.g. ideal alignment), movement and/or orientation, thereby reducing the risk of injury.

A thirteenth aspect of the invention is a wearable spinal alignment apparatus comprising the spinal alignment component according to the twelfth aspect, and a securement system to attach the apparatus to the torso of a wearer. The wearable spinal alignment apparatus may be a backpack.

Further optional features and advantages of the present invention are set out below.

### DISCLOSURE OF INVENTION

The spinal alignment component according to the first aspect of the invention is adapted for incorporation into a wearable spinal alignment apparatus. The spinal alignment component comprises a first rigid elongate member comprising a first distal end; and a second rigid elongate member comprising a second distal end; wherein the first rigid elongate member and the second rigid elongate member are independently movable relative to one another to adjust the displacement between the first distal end and the second distal end.

The component comprises separate first and second rigid elongate members which are independently movable relative to one another to adjust the displacement between the first distal end and the second distal end. In some embodiments, the component is adjustable into an extended configuration by moving one or both of the first and second rigid elongate members, wherein in the extended configuration the component comprises first, second and third contact portions arranged collinearly along the component and disposed to contact a wearer's occipital head, sacral spine and thoracic spine respectively when secured to the back of the wearer. In some embodiments, the component is adjustable into a retracted configuration by moving one or both of the first and second rigid elongate members, wherein in the retracted configuration the component comprises a third contact portion disposed to contact a wearer's thoracic spine when secured to the back of the wearer. In some embodiments, in the retracted configuration the component does not lie adjacent to or contact either of the wearer's occipital head or sacral spine.

One or both of the first and second rigid elongate members may be moved relative to one another (extended) to increase the displacement between the first distal end and the second distal end, or one or both of the first and second rigid elongate members may be moved relative to one another (retracted) to decrease the displacement between the first distal end and the second distal end. A device is thereby provided which is adjustable in a number of different ways to suit the needs of the user. The user may move (extend or retract) only one of the two rigid elongate members where required, or may move (extend or retract) both rigid elongate members.

In some cases the user may wish to move both of the two rigid elongate members to increase the displacement between the first distal end and the second distal end. The user may move both of the first and second rigid elongate members until the first contact portion on the first rigid elongate member is aligned with the user's occipital head and the second contact portion on the second rigid elongate member is aligned with the user's sacral spine, when the component is incorporated into wearable apparatus worn by the user. In this way, when the user wishes to adopt ideal alignment they ensure contact of the first contact portion with the occipital head, the second contact portion with the sacral spine and the third contact portion with the thoracic spine. Alternatively, the user may wish to adopt a configuration in which there is a reduced displacement between the first distal end and the second distal end. The user may move the first rigid elongate member until the first contact portion on the first rigid elongate member is aligned with the user's occipital head, and the user may move (retract) the second rigid elongate member to provide a reduced displacement between the first distal end and the second distal end. In this way, the second rigid elongate member is retracted, such that the user may, for example, sit down while using the device while still being able to ensure good alignment between the occipital head and thoracic spine, thereby reducing the risk of e.g. work related upper limb disorders (WRULD).

In other situations the user may move (retract) both of the two rigid elongate members to decrease the displacement between the first distal end and the second distal end. In this way the component adopts a compact configuration with retracted rigid elongate members, which facilitates transport of the component or apparatus into which it is incorporated. This is particularly advantageous when the component is incorporated into a bag or clothing and the user wishes to wear the bag or clothing without the component being visible to others. The user may then extend the first and second elongate members at a suitable point in time when they wish to use the bag or clothing for posture improvement.

The spinal alignment component is adapted for incorporation into a wearable spinal alignment apparatus. In other words, the spinal alignment component comprises a means to incorporate the component into a wearable spinal alignment apparatus such as a backpack, harness or clothing. In this way the component is specifically designed and adapted for use in a wearable spinal alignment apparatus to provide a device which the user may wear for posture improvement training. In this way a versatile device is provided which may find use in a variety of applications depending on the particular needs of the user or manufacturer. Incorporation of the component into a wearable spinal alignment apparatus may be carried out by a manufacturer, such that it is not intended for the component to be removed from wearable spinal alignment apparatus by the user during normal use of the apparatus. For example, the component may be stitched into a wearable spinal alignment apparatus, such as a bag or clothing, by the manufacturer. Thus in some embodiments, the component is irreversibly incorporated into a wearable spinal alignment apparatus. In this context, the term “irreversibly incorporated” pertains to securement of the component into the wearable spinal alignment apparatus in such a way that the component cannot be easily removed from the wearable spinal alignment apparatus without causing damage to the wearable spinal alignment apparatus.

In alternative embodiments, incorporation of the component into a wearable spinal alignment apparatus may be carried out by a user, such that the user themselves are able to choose the particular application for the component. In such embodiments, a wearable spinal alignment apparatus, such as a bag or clothing, may be provided which is complementary to the component, such that the component may be reversibly incorporated into the wearable spinal alignment apparatus. The wearable spinal alignment apparatus may be specifically adapted to receive the component, for example by comprising a cavity which has a complementary size and shape to the component. The component may therefore comprise means to reversibly secure the component in place.

In some embodiments, the component comprises means to secure each of the first rigid elongate member and second rigid elongate member in a predetermined specific position. Such means may permit each of the first rigid elongate member and second rigid elongate member to be fixed in one of a plurality of positions. In some embodiments, each of the first rigid elongate member and second rigid elongate member comprises means to secure the member in a first, fully retracted position and a second, fully extended position. In some embodiments, each of the first rigid elongate member and second rigid elongate member comprises means to secure the member in one or more positions intermediate the first, fully retracted position and second, fully extended position. In this way, the user may select positions for each of the first rigid elongate member and second rigid elongate member suitable for the intended use of the component. For example, depending on the size of the user (i.e., the distance between the user's thoracic spine and each of their occipital head and sacral spine), they may select a fully extended position, or intermediate extended position, for each of the first rigid elongate member and second rigid elongate member such that, when incorporated into a wearable spinal alignment apparatus and worn by the user, the first contact portion contacts the occipital head and the second contact portion contacts the sacral spine.

In some embodiments, the spinal alignment component further comprises a housing which receives each of the first rigid elongate member and second rigid elongate member, wherein each of the first rigid elongate member and second rigid elongate member are reversibly extendable from the housing. In some embodiments, each of the first distal end and the second distal end remain external to the housing when the first rigid elongate member and second rigid elongate member are fully retracted into the housing. Providing such a housing allows the members to be easily adjustable relative to one another by extending or retracting the members from or into the housing as required. Such a housing also provides a means to conveniently store or conceal the rigid elongate members when the component is being transported or is not being used for posture training. Providing a means to store the elongate members in this way also reduces the risk of damage to the elongate members, extending the lifetime of the device.

In some embodiments the housing has an elongate structure which is complementary to the elongate structure of the rigid elongate members. In some embodiments the housing has a primary axis which lies parallel with a primary axis of each of the first and second rigid elongate members.

In some embodiments, each of the respective first and second rigid elongate members are slidably received within the housing. In some embodiments, each of the respective first and second rigid elongate members are axially slidable within the housing. In this way the members may be retracted into or extended from the housing easily by sliding the members within the housing.

In some embodiments, the housing defines one or more channels configured to receive and guide the first and second rigid elongate members. In some embodiments, the housing defines a first channel configured to receive and guide the first rigid elongate member and a second channel configured to receive and guide the second rigid elongate member. In some embodiments the first and second channels are parallel. In some embodiments the first and second channels are separated by a partition wall within the housing. The partition wall may extend along the entire interior length of the housing. The first rigid elongate member may be slidably received by the first channel. The second rigid elongate member may be slidably received by the second channel.

In some embodiments, the members are slidable within the housing by providing complementary cross-sections of each member with a channel defined in the housing. In some embodiments the rigid elongate member and the channel adapted to receive and guide it comprise complementary surface structures to facilitate guiding of the rigid elongate member. These complementary structures may comprise a groove and a complementary rail which fits and slides within the groove.

In some embodiments, the housing defines a first channel configured to receive and guide the first rigid elongate member and a second channel configured to receive and guide the second rigid elongate member, wherein the first and second channels are parallel and separated by a partition wall within the housing, wherein a first end of the housing comprises a first end of the first channel and a first end of the second channel, and a second end of the housing comprises a second end of the first channel and a second end of the second channel, wherein the first end of the first channel and the second end of the second channel are open and able to slidably receive one of the first or second rigid elongate members, and the second end of the first channel and second end of the second channel are sealed to prevent passage of the first or second rigid elongate member. In this way, a compact means is provided to house independently extendable first and second rigid elongate members within the same housing but extendable in diametrically opposite directions.

In some embodiments, the housing defines a first channel configured to receive and guide the first rigid elongate member and a second channel configured to receive and guide the second rigid elongate member, wherein the first and second channels are parallel and separated by a partition wall within the housing, and wherein the housing further comprises a first end-cap covering both the first and second channels at one end of the housing and a second end-cap covering the both the first and second channels at the other end of the housing, wherein the first end-cap blocks the second channel and defines an aperture providing access to the first channel; and the second end-cap blocks the first channel and defines an aperture providing access to the second channel. In this way, the first elongate member may be received into the first channel at one end of the housing via the aperture in the first end-cap and may be guided by the channel to advance along the housing until the end of the first rigid member meets the second end-cap, which is arranged to block the exit of the first channel. Similarly, the second elongate member may be received into the second channel at one end of the housing via the aperture in the second end-cap and may be guided by the channel to advance along the housing until the end of the second rigid member meets the first end-cap, which is arranged to block the exit of the second channel. In this way the travel of the first and second rigid elongate members along the channels is limited to prevent over-retraction into the housing beyond a desired extent. Furthermore this provides a compact means to house independently extendable first and second rigid elongate members within the same housing but extendable in diametrically opposite directions.

In some embodiments, the first rigid elongate member is extendable from the housing by moving the first rigid elongate member in a first direction and the second rigid elongate member is extendable from the housing by moving the second rigid elongate member in a second direction, wherein the first direction is diametrically opposed to the second direction. In this way, the extension of both of the first and second rigid elongate members from the housing extends the length of the overall component to facilitate contact of the first rigid elongate member with the occipital head and the second rigid elongate member with the sacral spine.

In some embodiments, the housing is a rigid elongate housing defining a first channel and a second channel, the first channel being adapted to receive the first rigid elongate member and the second channel being adapted to receive the second rigid elongate member, wherein the respective first and second rigid elongate members are each slidably received within the housing. In some embodiments, the first channel and the second channel are parallel channels defined by the housing and separated by a partition wall.

In some embodiments, each of the first channel and the second channel extend along the entire axial length of the housing.

In some embodiments, the spinal alignment component comprises a friction fit or interference fit mechanism between the housing and each of the first rigid elongate member and second rigid elongate member. In some embodiments, the spinal alignment component comprises an incrementally adjustable friction fit or interference fit mechanism between the housing and each of the first rigid elongate member and second rigid elongate member. The friction fit mechanism may comprise a resilient protrusion on a first surface which cooperates with a linear array of teeth distributed along a second surface; wherein one of the first and second surface comprises an inner surface of the housing and the other of the first and second surface comprises an outer surface of the first or second rigid elongate member; such that the protrusion moves along the linear array of teeth as the rigid elongate member is moved relative to the housing, and interference between the resilient protrusion and one or more teeth from the linear array facilitates the temporary securement of the rigid elongate member in a specific position relative to the housing. In some embodiments, the interference is overcome by applying a sufficient level of force to the rigid elongate member in an axial direction thereby advancing the elongate member axially until the force is removed and interference between the resilient protrusion and a tooth on the housing secures the rigid elongate member in a new position. Such an interference mechanism provides a simple means to adjust the configuration of the component without the need to first actuate any trigger mechanisms such as buttons or levers. This provides fast adjustability which is desirable, for example, when the user is transitioning between exercises and wishes to quickly adjust the configuration of the component to suit the next exercise.

In this way, the component is easily adjustable by the user by the application of a small amount of force to overcome the friction between the housing and first or second rigid elongate member and move the member relative to the housing. Simultaneously, the force required to overcome the friction fit may be sufficient to prevent the inadvertent movement of a member relative to the housing, for example under the influence of gravity. In this way, once the positions of the first and second elongate members are set by the user, their positions are fixed during use, providing improved functionality of the device.

The spinal alignment component may be adapted for incorporation into a wearable spinal alignment apparatus by comprising attachment means for securement to a wearable component. The spinal alignment component may be adapted for incorporation into a wearable spinal alignment apparatus by including attachment means on the housing for securement to a wearable component. The attachment means may comprise one or more flanges extending outwardly from a main body of the housing. The one or more flanges may each comprise one or more apertures through which a securement means may be passed.

In some embodiments, the displacement between the first distal end and the second distal end is adjustable to correspond with the distance between the occipital head and the sacral spine of a user when the user is in ideal alignment.

In some embodiments, the component comprises sensing means, for example one or more sensors. In some embodiments the housing of the component comprises sensing means. In some embodiments one or both of the rigid elongate members of the component comprise sensing means. The sensing means may be adapted to measure the position of the component relative to a body part of a user, for example by providing proximity or pressure sensors to detect proximity to or contact with a body part. The sensing means may be adapted to measure the position or orientation of the component in space, for example by providing one or more sensors selected from an accelerometer, a gyroscope, a magnetometer, a three degrees of freedom (3DOF) tracker and a six degrees of freedom (6DOF) tracker.

In some embodiments, each of the first distal end and the second distal end comprise a sensing means, wherein the sensing means is adapted to sense contact or proximity between a contact portion and the wearer's body, and to generate sensor data, and a communication means, wherein the communication means is adapted to communicate the sensor data with one or more user devices.

In some embodiments, each of the first and second elongate components comprises a terminal region having an enlarged profile adjacent to the respective first distal end and second distal end. In some embodiments, the enlarged profile is provided by a protrusion of the terminal region extending laterally in one direction.

In some embodiments, the enlarged profile is provided by a protrusion of the terminal region extending laterally in one direction.

The enlarged profile facilitates close contact between the terminal regions and the occipital head or sacral spine of the wearer.

In some embodiments, the terminal region of each of the first and second elongate components comprises a soft or flexible material. In this way, a comfortable contact with the occipital head or sacral spine is provided so that the wearer can achieve ideal alignment without feeling pain or discomfort.

In some embodiments, the terminal region is a removable cap. In some embodiments, the terminal region is a removable cap made from a soft or flexible material. In some embodiments, the removable cap is push-fitted to the rigid elongate member. The removable cap may house a sensing means, wherein the sensing means is adapted to sense contact or proximity between a contact portion and the wearer's body, and to generate sensor data, and a communication means, wherein the communication means is adapted to communicate the sensor data with one or more user devices.

For example, the removable cap may comprise a Bluetooth transmitter adapted to transmit sensor data to associated hardware such as a mobile device, and the associated hardware may comprise suitable software, such as a computer program or app, to interpret the data and provide useful feedback to the user. In some embodiments, audio feedback may be provided to the user, for example through headphones. Thus the user may receive real-time information or feedback on their body position. The user may also receive instructions regarding what body movements may be necessary to correct body position to achieve ideal alignment, based on the sensor data received by the sensors in the component and transmitted to the associated hardware. As an example, should the user's occipital head lose contact with the terminal region (contact portion) of the first rigid elongate member, the sensor within the first rigid elongate member would detect this and send data concerning the lost contact, via Bluetooth, to the user's mobile device. An app installed on the user's mobile device may then notify the user, for example by visual or audio cues, of the lost contact, encouraging the user to tilt the head backwards to reinstate contact with the first rigid elongate member and achieve ideal alignment again.

The spinal alignment component may be a spinal alignment training component, in other words a component for use in posture training. In particular the component is for use during fitness or strength training, for example at a gym, to ensure optimal body positioning and movement to improve form and reduce the risk of injury.

A second aspect of the invention is a wearable spinal alignment apparatus comprising the spinal alignment component according to the first aspect, and a securement system to attach the apparatus to the torso of a wearer.

Such a spinal alignment apparatus facilitates the use of the spinal alignment component by a wearer to improve posture, for example while exercising or going about daily activities. The wearer may use the securement system to secure the spinal alignment component against their torso in contact with their thoracic spine, and extend one or both of the rigid elongate members such that they are adjacent one or both of their occipital head and sacral spine.

In some embodiments, the securement system comprises one or more attachment points adapted to attach to one or more straps. In some embodiments, the securement system comprises one or more straps connected to the spinal alignment component in such a way as to enable to component to be secured against the back of a wearer in contact with the wearer's thoracic spine.

The wearable spinal alignment apparatus may comprise a harness connected to the spinal alignment component according to the first aspect. The harness may comprise a securement system comprising left and right shoulder loops adapted to encircle the wearer's left and right shoulders. The harness may comprise at least one horizontal securement strap to pass around the wearer's lower torso or waist.

The wearable spinal alignment apparatus may comprise the spinal alignment component according to the first aspect, and an external housing, wherein the spinal alignment component is held within the external housing, the external housing being adapted for securement to the torso of a wearer by a securement system, and the external housing defining first and second apertures through which each of the first and second rigid elongate members respectively pass. The securement system may comprise one or more attachment points for straps. The securement system may comprise one or more straps.

In some embodiments, one or more cavities are defined between the spinal alignment component and an inner wall of the external housing.

In some embodiments, the spinal alignment component is fixedly attached to the external housing.

In some embodiments, the external housing surrounds the housing of the spinal alignment component and defines first and second apertures through which each of the first and second rigid elongate members respectively pass.

In some embodiments, the external housing comprises a hollow shell. In some embodiments, the external housing comprises a hollow shell defined by a first wall and a second wall, wherein the first and second walls are attached to one another around their respective peripheries. The first and second walls may be attached by stitching. In some embodiments, one or both of the first and second walls comprises a peripheral flange region adapted for attachment to the second of the walls. In some embodiments, the first and second apertures are each located either through one of the first and second wall or between the first and second wall.

The external housing may comprise flexible material. This provides a more comfortable surface against which a body part of a wearer may rest during use.

The external housing may comprise one or more elongate apertures providing access to one or more internal cavities, wherein the one or more internal cavities are defined between the external housing and the housing of the spinal alignment component. The one or more elongate apertures may be reversibly sealable by a closure means. In some embodiments, the closure means comprises a zip or buttons. In some embodiments, the one or more elongate apertures are located on a surface of the external housing which is adapted to contact part of the body of a wearer when the wearable spinal alignment apparatus is being work by the wearer. In this way, the one or more elongate apertures are concealed against the body of the wearer and not easily visible. This provides a discreet design and also reduces the risk of tampering or theft from the wearable spinal alignment apparatus during use. In some embodiments, two elongate apertures are provided which extend substantially parallel with the orientation of the spinal alignment component when the spinal alignment component is secured within the external housing. This provides access to cavities defined between the spinal alignment component and an inner wall of the external housing on either side of the component, along a substantial length of the cavities, improving the storage capability and functionality of the wearable spinal alignment apparatus.

The external housing is adapted for securement to the torso of a wearer by a securement system comprising one or more straps. In some embodiments, the external housing comprises one or more attachment points for the attachment of a strap. The attachment point may comprise a buckle or clasp. In some embodiments, the external housing comprise a plurality of shoulder strap attachment points and a plurality of waist strap attachment points.

In some embodiments a cavity within the external housing is defined between a first wall and a second wall, the first and second walls defining the outer surface of the external housing. The first wall may form the outer surface of a portion of the external housing intended to or adapted to contact a body part of a user when attached to the user's torso during use, and the second wall may form the outer surface of a portion of the external housing not intended to or adapted to contact a body part of a user. In some embodiments the first wall comprises a flexible or “soft” material. In some embodiments the first wall comprises a material selected from foam and fabric. In some embodiments the first wall comprises a layer of foam and a layer of fabric. Such flexible or “soft” material provide a comfortable surface against which the body part (e.g. back) of the user may contact during use. In some embodiments the second wall comprises a rigid or semi-rigid material. Non limiting examples of such materials include EVA foam and rigid or semi-rigid plastics materials. In this way, the external housing keeps its shape, preventing damage to any contents within the cavity. The combination of the first and second walls provides a resilient external housing which keeps its shape while being comfortable to wear. The first wall and second wall may be fixed together, for example by stitching. One or more of the first and second wall may comprise an external flange portion to facilitate fixing of the first wall to the second wall.

In some embodiments, the one or more elongate apertures described above may be present in the first wall of the external housing, thereby being located against the wearer's back when the apparatus is worn by the wearer.

In some embodiments, the first wall of the external housing comprises one or more protrusions, each protrusion comprising an attachment point for the attachment of a strap.

In this way, the attachment points are located on a flexible protrusion which is an extension of the first wall, thereby being able to bend and confirm with the contours of the wearer's body when the straps are secured, thereby improving comfort.

In some embodiments, the spinal alignment component is fixedly attached within the external housing by stitching. In some embodiments, the spinal alignment component is secured in place within the external housing due to the first and second rigid elongate members being located within the first and second apertures. In such embodiments, further securement of the component to the external housing may not be necessary. In some embodiments, the size and shape of the first and second apertures corresponds with the cross-section of the first and second rigid elongate members respectively, thereby providing a snug fit and preventing or reducing lateral movement of the component within the external housing.

In some embodiments the wearable spinal alignment apparatus comprises the spinal alignment component according to the first aspect, and a backpack, wherein the spinal alignment component is fixedly attached to the backpack.

In some embodiments, the spinal alignment component is fixedly attached to the backpack by stitching.

A third aspect of the invention is the use of the spinal alignment component according to the first aspect in a wearable spinal alignment apparatus.

A fourth aspect of the invention is the use of the spinal alignment component according to the first aspect in the manufacture of a wearable spinal alignment apparatus.

A fifth aspect of the invention is the use of the spinal alignment component according to the first aspect to improve or correct the posture of a user.

A sixth aspect of the invention is the use of the spinal alignment component according to the first aspect in a wearable alignment device or apparatus.

A seventh aspect of the invention is a wearable alignment device, comprising a rigid elongate member comprising first, second and third contact portions disposed along its length, such that when the device is worn by a wearer the first, second and third contact portions are arranged collinearly and disposed to contact the wearer's occipital head, sacral spine and thoracic spine respectively; and a securement system comprising left and right shoulder loops adapted to encircle the wearer's left and right shoulders, and at least one horizontal securement strap adapted to connect the left and right shoulder loops across the wearer's front; wherein when the device is worn by a wearer the securement system is disposed to secure the third contact portion in contact with the thoracic spine, and the first and second contact portions remain movable away from the occipital head and sacral spine respectively.

In some embodiments, the seventh aspect of the invention is a wearable alignment device, comprising a spinal alignment component according to the first aspect of the invention, comprising first, second and third contact portions disposed along its length, such that when the device is worn by a wearer the first, second and third contact portions are arranged collinearly and disposed to contact the wearer's occipital head, sacral spine and thoracic spine respectively; and a securement system comprising left and right shoulder loops adapted to encircle the wearer's left and right shoulders, and at least one horizontal securement strap adapted to connect the left and right shoulder loops across the wearer's front; wherein when the device is worn by a wearer the securement system is disposed to secure the third contact portion in contact with the thoracic spine, and the first and second contact portions remain movable away from the occipital head and sacral spine respectively.

When the device of the seventh aspect is worn by a wearer the first, second and third contact portions are arranged collinearly and disposed to contact the wearer's occipital head, sacral spine and thoracic spine respectively. In other words, when the wearer's occipital head, sacral spine and thoracic spine are placed in contact with the first, second and third contact portions of the elongate member respectively, the wearer will be in ideal alignment.

When the device of the seventh aspect is worn by a wearer, the third contact portion of the rigid elongate member is held in contact with the thoracic spine of the wearer by the securement system. The securement system is connected to the rigid elongate member in such a way that when the securement system is used to attach the device to a wearer, the rigid elongate member is disposed to secure the third contact portion in contact with the thoracic spine, and the first and second contact portions remain movable away from the occipital head and sacral spine respectively.

Herein, “thoracic spine” refers to the portion of length of the spine which comprises vertebrae T2 to T7, preferably vertebrae T4 to T6. Herein, “sacral spine” refers to the portion of length of the spine which comprises vertebrae S2 to S5, preferably vertebrae S4 to S5. Herein, “occipital head” refers to the back of the head of the wearer, in particular including the upper part of the occipital bone.

In some embodiments the wearable alignment device does not comprise any means to secure the device to the wearer's waist. In this way, the wearer is able to break the ideal alignment by bending at the waist when needed, for example when moving between exercises or preparing weights. Nevertheless, the securement of the device to the wearer's thoracic spine means that the wearer is still able to adopt ideal alignment when required by sensing contact of the spine with all three contact portions of the elongate member. This also minimises the restriction of movement experienced by the wearer, so everyday activities and exercise are not hindered. For example, in some embodiments the wearable alignment device does not comprise a securement strap arranged to encircle the wearer's waist.

In some embodiments the wearable alignment device does not comprise any means to secure the device to the wearer's head. In this way, the wearer is able to break the ideal alignment by moving the neck or turning the head when needed, for example when moving between exercises or running. Nevertheless, the securement of the device to the wearer's thoracic spine means that the wearer is still able to adopt ideal alignment when required by sensing contact of the spine with all three contact portions of the elongate member. This also minimises the restriction of movement experienced by the wearer, so everyday activities and exercise are not hindered. For example, in some embodiments the wearable alignment device does not comprise a securement strap arranged to encircle the wearer's head.

In some embodiments the wearable alignment device does not comprise either a means to secure the device to the wearer's waist or a means to secure the device to the wearer's head. The wearable alignment device is thereby secured to the wearer without any forcible contact between the elongate member and the occipital head and sacral spine (i.e. the two peripheral points on the ideal alignment line). The elongate member is free to move away from the occipital head and the sacral spine to some extent, permitting a wider range of movements for the wearer when necessary. The device is therefore more versatile, can be worn for longer periods (for example, between exercises where ideal alignment is not necessary) and does not force the wearer into ideal alignment providing improved muscle memory because the wearer is effectively mentally and physically adapting to maintain ideal alignment and correct posture; this stops the wearer becoming reliant on an alignment or posture training device.

In some embodiments the left and right shoulder loops and horizontal securement strap(s) are constructed of a flexible material, wherein the flexible material is a fabric. In some embodiments the fabric is elasticated and in some such embodiments the fabric comprises elastane. In some embodiments the fabric is a low density material, in some such embodiments the fabric is a breathable or water permeable material. In some embodiments the left and right should loops are constructed of neoprene. In some embodiments the horizontal securement strap(s) are constructed of neoprene. In some embodiments the left and right should loops and the horizontal securement strap(s) are constructed of neoprene.

The securement system secures the third contact portion firmly in contact with the thoracic spine, even when the wearer is breathing heavily or engaged in movements involving flexion of the spine. The shoulder loops and horizontal securement strap(s) also increase the wearer's comfort during a broad range of everyday activities and exercise.

The use of shoulder loops and one or more horizontal securement straps provides a secure instalment of the device on the wearer since unintended vertical movement of the device along the body of the wearer is prevented, without the need to secure the device with uncomfortable tightness. By contrast, a device which includes only one or more straps horizontally encircling the torso requires tight attachment to the wearer to prevent the device falling down the wearer's torso during use. Such tight attachment is uncomfortable, restricts breathing and movement, and may cause pain and/or injury.

The shoulder loops may be directly attached to the rigid elongate member or may be attached to a separate body, for example a body of material, which is in turn attached to the rigid elongate member. The shoulder loops may be formed from a single length of material or multiple lengths of material which are separately connected to the rigid elongate member or to a separate body which is in turn attached to the rigid elongate member.

In some embodiments the shoulder loops have a varying width along their length, wherein the width is greatest for the portion in contact with the top of the shoulder and least for the portion between the arm and torso. In some embodiments the shoulder loops comprise foam padding. This serves to reduce the pressure of the shoulder loops on the wearer's body while minimally restricting the wearer's movement.

The shoulder loops are formed to hold the third contact portion in contact with the thoracic spine, and to minimise vertical and horizontal displacement of the contact portions, in particular when the wearer is involved in exercise and everyday activities that involve vertical movement, such as running.

The securement system further comprises at least one horizontal securement strap which is adapted to connect the left and right shoulder loops. In some embodiments this is adapted to fasten between the left and right shoulder loops. In some embodiments the strap is elasticated. In some embodiments the strap comprises foam padding. This serves to reduce the pressure of the straps on the wearer's body while minimally restricting the wearer's movement.

Such a strap assists in holding the third contact portion in contact with the thoracic spine, and minimises vertical and horizontal displacement of the contact portions, in particular when the wearer is involved in exercise and everyday activities that involve horizontal movement, such as cross training or weight lifting. The horizontal securement strap(s) also helps to keep the position of the shoulder loops fixed during use, ensuring that the rigid elongate member remains in the correct position for facilitating ideal alignment.

The combination of shoulder loops and horizontal securement strap(s) provides very secure contact between the third contact portion of the elongate member and the thoracic spine, while still permitting movement of the occipital head and sacral spine of the wearer relative to the elongate member.

In some embodiments the securement system comprises one or more fastening means, wherein one or more of the shoulder loops or horizontal securement straps are reversibly detachable by the fastening means. In some embodiments at least one of the horizontal securement straps are attached to the left and right shoulder loops and adapted to fasten between the left and right shoulder loops via a fastening means, thereby reversibly connecting the left and right shoulder loops. In some embodiments the two shoulder loops, which in use encircle the wearer's left and right shoulders further comprises fastening means. In some embodiments one or more of the shoulder loops and horizontal securement strap(s) comprises fastening means.

The particular form of the fastening means is not limited and many suitable means for temporarily fastening two lengths of strap together are well-known to the skilled person. Preferably, the securement straps are adapted to be temporarily fastened together by fastening means, i.e. they comprise a suitable adaptation rather than simply requiring tying into a knot by the wearer to secure.

In some embodiments the fastening means comprises a hook and loop closure. In some embodiments the fastening means comprises a buckle and clasp fastener.

The fastening means facilitates easy attachment and detachment of the device to the body by the wearer, without the need for assistance by a third party. The wearer may select combinations of the shoulder loops and horizontal securement straps for use in different everyday activities or exercises. This serves to provide a more versatile device for use during a variety of activities. For example, the wearer may select only shoulder loops while walking or setting up weight lifting equipment, but then may additionally engage the horizontal securement straps while running or carrying out weight lifting.

In some embodiments the securement system comprises one or more adjustment means, wherein the adjustment means are operable to reversibly adjust the length of one or more of the shoulder loops or horizontal securement straps. In some embodiments the left and right shoulder loops each further comprise adjustment means. In some embodiments at least one of the horizontal securement straps further comprise adjustment means. In some embodiments, one or more of the shoulder loops and horizontal securement straps comprise adjustment means.

In some embodiments the adjustment means comprises a hook and loop closure wherein the degree of overlap of the hook section and loop section will adjust the length of the securement strap or shoulder loop. In some embodiments the adjustment means comprises a buckle or clasp fastening means. In some embodiments the adjustment means comprise a friction based ratchet system.

The particular form of the adjustment means is not limited and many suitable means for adjusting the length of a strap are well-known to the skilled person. In some embodiments the adjustment means comprise means to temporarily hold or fix the strap at a particular length after adjustment.

The adjustment means allows the device to be used by a broader size range of wearers. The adjustment means allows the wearer to modulate the pressure from the third contact portion onto the thoracic spine, modulate the pressure of the securement straps or shoulder loops on the shoulders and torso, and modulate the restraint provided by the device. This results in the device being more comfortable and useable during a broad range of everyday activities and exercise.

The rigid elongate member must be long enough to facilitate contact with the wearer's occipital head, sacral spine and thoracic spine. It must also have sufficient rigidity, or stiffness, such that during use the three contact portions remain substantially collinear.

Herein, “substantially collinear” indicates that under the normal forces which can be expected to be exerted on the elongate member by a wearer during use, the elongate member will deform from linear by a maximum perpendicular distance of 5 cm, for example a maximum of 4 cm, a maximum of 3 cm, a maximum of 2 cm, or a maximum of 1 cm (where the perpendicular distance is measured between the position of the end of the elongate member when deformed and its position when linear).

The particular material and form of the elongate rigid member is not limited and many suitable materials and shapes are well-known to the skilled person. In some embodiments the elongate member is cylindrical. In some embodiments the elongate member is hollow. In some embodiments the elongate member is cylindrical and hollow. In certain embodiments the elongate member is made from a suitable metal, such as aluminium. In certain embodiments the elongate member is made from a suitable rigid plastic.

The cylindrical shape is complementary to the back of the wearer and is adapted to fit between the shoulder blades of the wearer. A cylinder also provides a singular contact line along the elongate member, ensuring accurate contact with the back of the wearer and accurate tactile feedback (compared to e.g. a planar elongate member which would provide a range of contact points at each vertical position). This construction is also low weight, which minimises the restriction of movement experienced by the wearer, so everyday activities and exercise are not hindered.

In some embodiments the elongate member is of a length such that in use the rigid elongate member does not protrude below the wearer's waist. In some embodiments the elongate member is of a length such that in use the rigid elongate member does not protrude above the wearer's head. Such a construction prevents the peripheral ends of the elongate member hindering the movement of the wearer and prevents the elongate member being displaced through contact with extraneous objects during everyday activities and exercise, for example, running under low hanging objects or sitting on an exercise bench.

In some embodiments the rigid elongate member has a fixed length, and a wearer chooses a size of device such that the elongate member is of a length such that in use the rigid elongate member does not protrude below the wearer's waist and/or the elongate member is of a length such that in use the rigid elongate member does not protrude above the wearer's head.

In some embodiments the rigid elongate member has a variable length, and a wearer may choose the length of the elongate member so that it does not protrude below the wearer's waist and/or does not protrude above the wearer's head.

In some embodiments the elongate member further comprises at least one extendable elongate section adapted to reversibly adjust the length of the elongate member, wherein the extendable elongate section is secured rigid and substantially collinear to the main body of the elongate member. In some embodiments the elongate member comprises two extendable elongate sections. In some embodiments the extendable elongate section comprises telescopic concentric tubular sections. In some embodiments the extendable elongate section comprises a plurality of detachable sections. In some embodiments the extendable elongate section comprises a plurality of detachable sections linked by a folding mechanism.

In some embodiments the extendable elongate section is secured rigidly and substantially collinear to the main body of the elongate member by at least one securing clip. In some embodiments the securing clip is a friction lock. In some embodiments the friction lock is a flip lock. In some embodiments the friction lock is a twist lock.

The variable length elongate member allows a single device to be used by a broader size range of wearers, thereby reducing the costs associated with providing multiple sizes of device and facilitating transfer of the device between wearers (e.g. for a shared device at, for example, a gym). This also enables the wearer to finely adjust the length of the elongate member for optimum fit, compared to the large jumps in elongate member length between different sized fixed length elongate member devices.

In some embodiments the securement system is incorporated into a bag. The bag may be a rucksack. In some embodiments a bag may be reversibly attached to the alignment device. In some embodiments the securement system is incorporated into clothing. The clothing may comprise a vest or gilet. This enables the device to be used easily during everyday activities and makes the device more aesthetically pleasing. The device is provided with additional useful functions, such as the ability to transport personal items.

In some embodiments the wearable alignment device comprises a sensor. In some embodiments the sensor is configured to generate sensor data. In some embodiments the sensor is configured to sense contact between a contact portion and the wearer's body. In some embodiments the sensor is configured to sense the orientation of the rigid elongate member, for example the orientation relative to the vertical. This is important because the preferred orientation when in ideal alignment is vertical, i.e. a position in which the line which passes through the apex of the sacral spine, the apex of the thoracic spine and the back of the occipital head is parallel with the vertical. A person adopting this position places the minimum level of strain on the back and the risk of injury is reduced.

In some embodiments the sensor is disposed about one or more of the contact portions along the elongate member, wherein the first, second and third contact portions have an optional first, second and third sensing means respectively. In some embodiments the first contact portion has a sensor. In some embodiments the first and second contact portions have a first and second sensor respectively. In some embodiments the first, second and third contact portions have a first, second and third sensor respectively.

In some embodiments the sensor is a mechanical force sensor, wherein the mechanical sensor comprises a tactile sensor, a force sensor or a load cell. In some embodiments the tactile sensor, force sensor or load cell comprise a piezo resistive sensor, a piezoelectric sensor, a capacitive sensor, an elastoresistive sensor, force sensing resistor, a pressure sensor array, a hydraulic load cell, a pneumatic load cell, or a capacitive load cell. The mechanical force sensor is adapted to detect the mechanical force acting upon it, and may distinguish the mechanical force when the wearer's body is in contact with and not in contact with the contact portion.

In some embodiments the sensor is a proximity sensor, wherein the proximity sensor is a capacitive proximity sensor, inductive proximity sensor, photoelectric proximity sensor, optical proximity sensor, or an ultrasonic proximity sensor. The proximity sensor is adapted to detect the distance between the wearer's body and the contact portion of the device, wherein if the distance is zero the wearer's body is contacting the contact portion and if the distance is greater than zero the wearer's body is not contacting the contact portion.

In some embodiments the sensor is an accelerometer or an inclinometer, adapted to sense the orientation of the rigid elongate member relative to vertical. This provides the device with the ability to indicate when the wearer is vertically aligned, for example, when the first, second and third (or the occipital head, sacral spine and thoracic spine respectively) are vertically collinear.

In some embodiments, the device comprises a first, second and third sensor at the first, second and third contact portions respectively, and a sensor adapted to sense the orientation of the rigid elongate member. In this way, the device is able to sense whether contact is made between the wearer and each of the first, second and third contact portions, and also sense the orientation of the rigid elongate member, e.g. so that the wearer can ensure a vertical orientation to reduce strain on the back.

In some embodiments the sensor data is categorical, wherein a first datum indicates contact between the wearer's body and the contact portion and a second datum indicates no contact between the wearer's body and the contact portion. In some embodiments the sensor data is continuous, wherein the force or distance indicates contact or no contact between the wearer's body and the contact portion.

The particular form of the sensor is not limited and many suitable means for sensing contact between a contact portion and the wearer's body, or sensing the orientation of the rigid elongate member, and generating sensor data, are well-known to the skilled person.

In some embodiments the wearable alignment device comprises a communication means, wherein the communication means is configured to communicate the sensor data with one or more user devices. In some embodiments the user device comprises any of a smart phone, a laptop computer, a smart television, a monitor, a tablet, a smart watch, smart eyewear, smart headphones or a computer. In some embodiments the communication means is configured to communicate the sensor data wirelessly.

In some embodiments the user device is configured to convert the sensor data into usable feedback, which guides the wearer to achieve ideal alignment. In some embodiments the user device is configured to convert the sensor data into useable feedback via an application. This provides the device with the ability to indicate when the wearer is contacting any of the first, second or third contact portions (with their occipital head, sacral spine or thoracic spine respectively) and is therefore in ideal alignment.

In some embodiments the feedback comprises kinaesthetic feedback, haptic feedback, or tactile feedback. In some embodiments the feedback comprises audio feedback, wherein the audio feedback comprises a variable spatial location, pitch, duration, volume, timbre of sound or verbal instruction. In some embodiments the feedback comprises visual feedback, wherein the visual feedback comprises signs, text, typography, animations, colours, lights, photographs, illustrations, diagrams or videos.

In some embodiments the wearable alignment device comprises a memory, configured to store the sensor data.

A twelfth aspect of the invention is a spinal alignment component adapted for incorporation into a wearable spinal alignment apparatus, wherein the spinal alignment component comprises sensing means to detect one or more of movement, position and orientation of a wearer during use of the component.

The sensing means may comprise one or more sensors. The sensing means may comprise one or more position/orientation sensors configured to measure a position or orientation of the component. The component may further comprise a controller configured to determine the position or orientation of the component based on the measured position or orientation. The one or more sensors may each be selected from an accelerometer, a gyroscope and/or a magnetometer. The sensors may be inertial measuring unit (IMUs). Specifically, the sensors may each comprise one or more accelerometers and one or more gyroscopes to measure the accelerations and angular velocities of the respective sensors (and therefore the respective body parts). The sensors may comprise an accelerometer and a gyroscope, wherein the accelerometer and the gyroscope are orthogonally mounted in the sensor. The sensors may be micro-electro-mechanical systems (MEMs) sensors, for example. The sensors may be three degrees of freedom (3DOF) trackers. In other embodiments, the sensors may be six degrees of freedom (6DOF) trackers.

The component according to the twelfth aspect may comprise a rigid elongate member, for example the rigid elongate member as described above in the context of the seventh aspect. The rigid elongate member may comprise first and second sensors. In some embodiments the first sensor is located at one axial extremity of the elongate member and the second sensor is located at the other axial extremity. The first and second sensors may each comprise one or more accelerometers and one or more gyroscopes to measure the accelerations and angular velocities of the respective sensors (and therefore the respective body parts associated with said sensors).

The component according to the twelfth aspect may have the structure of the component according to the first aspect, provided that it also comprises sensing means to detect one or more of movement, position and orientation of a wearer during use of the component. Thus the component may also comprise first and second rigid elongate member. In some embodiments the housing of the component comprises sensing means. In some embodiments the housing of the component comprises first and second sensors. In this way consistent and useful data on the position, movement or orientation of the wearer may be gathered by the component regardless of the configuration of the rigid elongate members. This allows the component to provide the user with useful information on their position, movement or orientation even when the rigid elongate members are in a retracted configuration, for example information relating to whether the wearer is in ideal alignment.

A thirteenth aspect of the invention is a wearable spinal alignment apparatus comprising the spinal alignment component according to the twelfth aspect, and a securement system to attach the apparatus to the torso of a wearer. The wearable spinal alignment apparatus may be a backpack.

The wearable spinal alignment apparatus of the thirteenth aspect provides a means for the wearer to monitor information such as the position, movement or orientation of one or more body parts and adapt their position, movement or orientation accordingly to achieve a more optimal condition.

Although the invention has been explained in relations to its preferred embodiments as mentioned above, it is to be understood that many other possible modifications and variations can be made without departing from the scope of the present invention. It is, therefore, contemplated that the appended claim or claims will cover such modifications and variations that fall within the scope of the invention.

### DESCRIPTION OF THE DRAWINGS

FIG. 1 shows (a) a side elevation and (b) a rear elevation of one embodiment of a spinal alignment component according to the invention.

FIG. 2 shows perspective views of a spinal alignment component according to the invention (a) in a fully retracted configuration, and (b) in a fully extended configuration.

FIG. 3 shows (a) an exploded view of a portion of one embodiment of a spinal alignment component according to the invention, and (b) a cross-sectional view of the housing of a spinal alignment component according to the invention.

FIG. 4 shows (a) a side elevation, (b) a front elevation, and (c) a perspective view of a first embodiment of a wearable spinal alignment apparatus according to the invention, incorporating the spinal alignment component depicted in FIGS. 1-3.

FIG. 5 shows an exploded view of the embodiment of a wearable spinal alignment apparatus from FIG. 4.

FIG. 6 shows (a) a side elevation, (b) a front elevation, and (c) a perspective view of a second embodiment of a wearable spinal alignment apparatus according to the invention, incorporating the spinal alignment component depicted in FIGS. 1-3.

FIG. 7 shows an exploded view of the embodiment of a wearable spinal alignment apparatus from FIG. 6.

FIG. 8 shows a third embodiment of a wearable spinal alignment apparatus according to the invention, incorporating the spinal alignment component depicted in FIGS. 1-3 within a backpack, in (a) extended configuration, rear elevation, (b) extended configuration, side elevation, and (c) retracted configuration, side elevation.

FIG. 9 shows a fourth embodiment of a wearable spinal alignment apparatus according to the invention, incorporating the spinal alignment component depicted in FIGS. 1-3 within a harness, in (a) front elevation, (b) extended configuration, rear elevation, (c) extended configuration, side elevation, and (d) retracted configuration, side elevation.

FIG. 10 is a perspective view of an embodiment of a wearable alignment device.

FIG. 11 is a front view of an embodiment of a wearable alignment device.

FIG. 12 is a perspective view of a wearer, wearing an embodiment of the wearable alignment device.

FIG. 13a is a side view of a wearer positioned in ideal alignment, wearing an embodiment of a wearable alignment device.

FIG. 13b is a side view of a wearer not positioned in ideal alignment, wearing an embodiment of the wearable alignment device.

FIG. 14 is a rear view of another embodiment of a wearable alignment device.

### DETAILED DESCRIPTION OF THE INVENTION

In the following description, like features in the drawings are given like reference numerals.

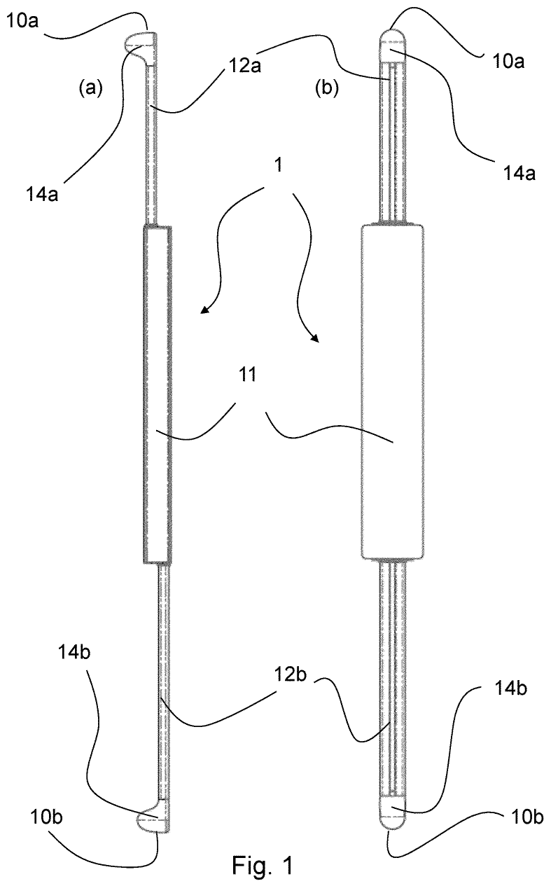

FIG. 1 shows a side elevation (a) and rear elevation (b) of a first embodiment of a spinal alignment component 1 according to the invention.

The spinal alignment component is made up of an elongate housing 11 made of rigid plastics material which independently receives and guides two rigid elongate members 12a and 12b. First rigid elongate member 12a extends upwardly (as seen in FIG. 1) out of the housing 11 through a first aperture (not shown) and second rigid elongate member 12b extends downwardly (as seen in FIG. 1) out of the housing 11 through a second aperture (not shown). Each of the first and second rigid elongate members 12a, 12b are slidably engaged with the housing 11 within respective internal channels defined by the housing 11. FIG. 1 shows the component in a fully extended configuration, with each of the first and second rigid elongate members 12a, 12b extended out of the housing 11 to the greatest extent possible. Further extension may be prevented by a detent mechanism within the component or may simply lead to complete removal of the rigid elongate member from the component, in which case the component could no longer fulfil its function until the user returns the elongate member to the housing. The first rigid elongate member 12a has a first distal end 10a which is the terminal portion of the first elongate member furthest from the housing 11, and the second rigid elongate member 12b has a second distal end 10b which is the terminal portion of the second elongate member furthest from the housing 11. In this fully extended configuration the displacement between the first distal end 10a and the second distal end 10b takes a maximum value. The first and second rigid elongate members and independently movable into and out of the housing by sliding along the respective channels provided in the housing.

The first and second rigid elongate members 12a, 12b are each made from aluminium in the embodiments shown.

An adjustable interference fit (described in more detail below) between the housing 11 and the respective first rigid elongate member 12a and second rigid elongate member 12b ensures that the rigid elongate members 12a, 12b maintain their chosen position within the channel of the housing and can be adjusted to select one of a plurality of available axial positions.

The first rigid elongate member 12a includes a terminal region which includes a first cap 14a which includes a lateral protrusion which extends laterally outwards from the rigid elongate member. An equivalent cap 14b lies at the terminal region of the second rigid elongate member 12b. The caps 14a, 14b each house sensor equipment including a gyroscope and Bluetooth connectivity hardware (not shown). The caps 14a, 14b are each formed from moulded soft, flexible plastic material or neoprene, thereby providing a comfortable contact point for the occipital head and sacral spine, respectively, of a user. The protrusion facilitates contact with the relevant body part of the user.

Each of the first and second rigid elongate members include axially oriented rails along their length which are complementary to axially oriented grooves within the channels of the housing 11, providing a secure fit between the elongate members and he housing (as described in more detail below).

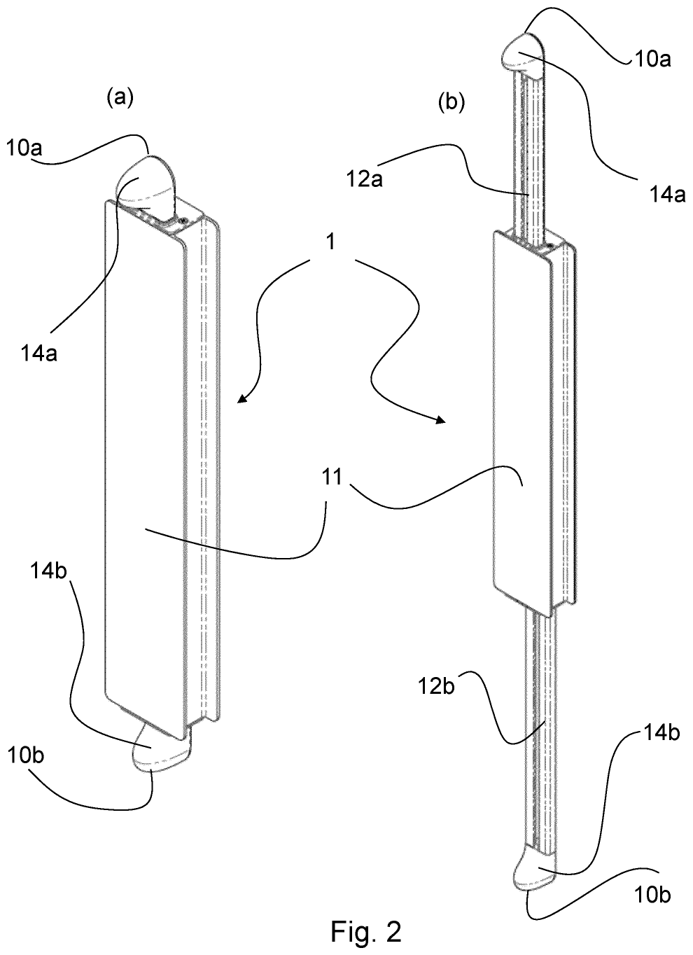

FIG. 2 shows perspective views of the same embodiment of the spinal alignment component shown in FIG. 1. In FIG. 2(a), the component is shown in its fully retracted configuration. In FIG. 2(a), the component is shown in its fully extended configuration. In the fully retracted configuration shown in FIG. 2(a), both rigid elongate members 12a and 12b have been pushed into the housing 11 until fully retracted, such that the displacement between the first distal end 10a and the second distal end 10b takes a minimum value. In the fully extended configuration shown in FIG. 2(b), both rigid elongate members 12a and 12b have been pulled from the housing 11 until fully extended, such that the displacement between the first distal end 10a and the second distal end 10b takes a maximum value. The retracted configuration shown in FIG. 2(a) may be suitable for transport or storage of the component. The extended configuration shown in FIG. 2(b) may be suitable when the component is in use as part of a wearable alignment device, as described in more detail below.

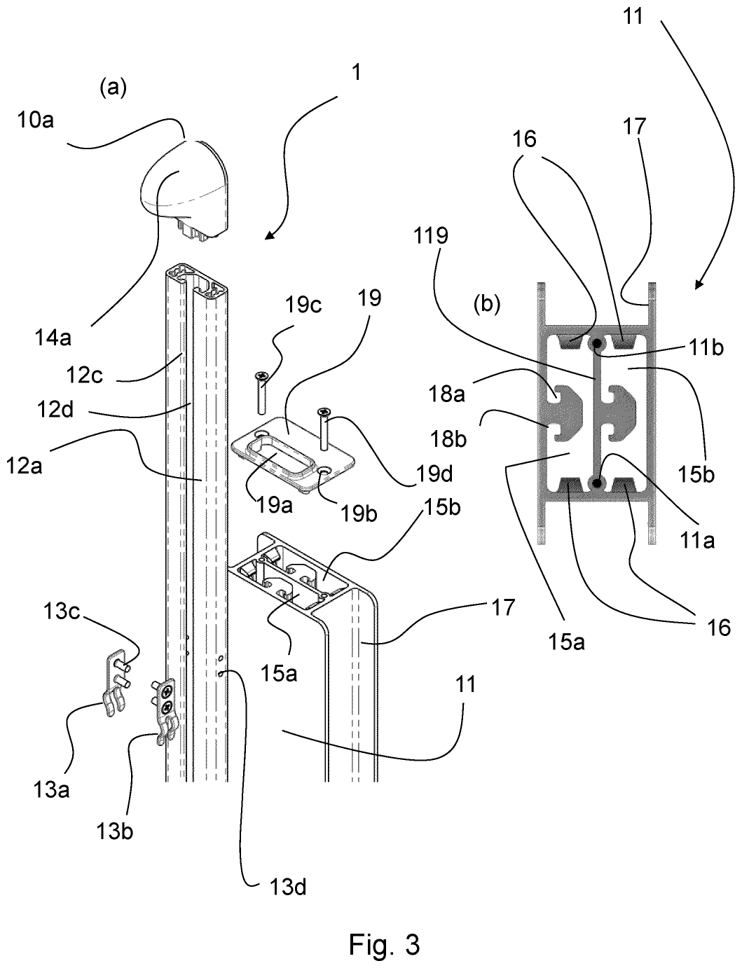

FIG. 3(a) shows an exploded view of a portion of the first embodiment of the spinal alignment component 1, showing each component in more detail. FIG. 3(b) shows a lateral cross-section of the housing 11.

First rigid elongate member 12a includes two resilient protrusions which are formed from resilient metal. Each of the resilient protrusions 13a, 13b includes a planar region which defines two apertures and a non-planar region which includes two leg portions. The resilient protrusions are attached to opposing outwardly facing edge surfaces of the first rigid elongate member 12a by screws 13c (of which only one is labelled in FIG. 3(a)) which pass through corresponding apertures in the resilient protrusion and engage with threaded cavities 13d (of which only one is labelled in FIG. 3(a)) in the first rigid elongate member 12a. When the resilient protrusion is attached to the first rigid elongate member the planar region lies flush with the outer surface of the first rigid elongate member and the non-planar region protrudes from the outer surface of the first rigid elongate member.

The cap 14a is attached to the rigid elongate member 12a by a push-fit. A friction fit is provided between a protrusion from the lower portion of the cap 14a and the rigid elongate member 12a.

The housing 11 defines axially extending first channel 15a and second channel 15b which extend along the entire length of the housing and are open at either end of the housing (such that each channel provides a passage or bore extending axially through the housing). Each channel includes a linear array of teeth 16 arranged along two opposing inner walls of the channel. Each resilient protrusion on the surface of the first rigid elongate member 12a interferes with a respective linear array of teeth 16 which lie along the internal surface of the channel 15a. This provides a plurality of discrete axial positions at which the first rigid elongate member 12a may be located within the channel 15a due to interference between the resilient protrusion 13a, 13b and the array of teeth 16.

A housing end-cap 19 is attached to the end of the housing 11 by passing screws 19c and 19d through corresponding apertures 116a and 19b and into threaded cavities 11a, 11b in the body of the housing 11. The housing end-cap 19 defines an aperture 19a whose dimensions correspond with the dimensions of the first rigid elongate member 12a, such that the rigid elongate member 12a is slidably received by the aperture 19a with a snug fit. When the housing end-cap 19 is fixed in place on the housing 12a, the opening of the channel 15a is accessible through the aperture 19a, however the opening of the channel 15a is blanked-off. This blanked-off end of the channel 15b provides a stop for the second rigid elongate member 12b when retracted into the housing 11 from the other end (not shown). The second rigid elongate member is pushed into the channel 15b until the end of the second rigid elongate member 12b meets the housing end-cap 19 which covers the end of the channel 15b, which provides a stop to prevent further retraction of the second rigid elongate member into the housing 11.

An equivalent housing end-cap (not shown) is installed on the other end of the housing 11, but in a reverse orientation, i.e. the aperture in the housing end-cap permits access to the entrance of the channel 15b but blanks off the channel 15a. Thus first rigid elongate member 12a will advance inwards along the channel 15a when pushed into the housing until the end of the first rigid elongate member 12a meets the housing end-cap (not shown) whereby any further movement of the first rigid elongate member into the housing is prevented.

FIG. 3(b) provides a cross-sectional view of the housing 11 in which the two parallel channels 15a and 15b can clearly be seen. The channels are each of a substantially C-shaped cross-section which complements the substantially C-shaped cross section of the two rigid elongate members 12a, 12b. The channels 15a, 15b are separated by a partition wall 119. Parallel rails 12c and 12d on the first rigid elongate member 12a fit within complementary grooves 18a, 18b in the wall of the channel 15a, providing a secure fit between the member 12a and the channel 15a an facilitating the sliding movement of the member 12a within and along the channel 15a.

The housing 11 comprises four laterally extending flanges 17 (only one of which is labelled in FIG. 3 for clarity) which provide a means to fixedly attach the housing 11 into a wearable spinal alignment apparatus. For example, the flanges 17 may fit into complementary pockets within a bag or clothing. The flanges 17 may include one or more apertures (not shown) to provide an attachment point for securing into wearable spinal alignment apparatus.

The housing may be manufactured by extruding a plastic material through an appropriately shaped die.

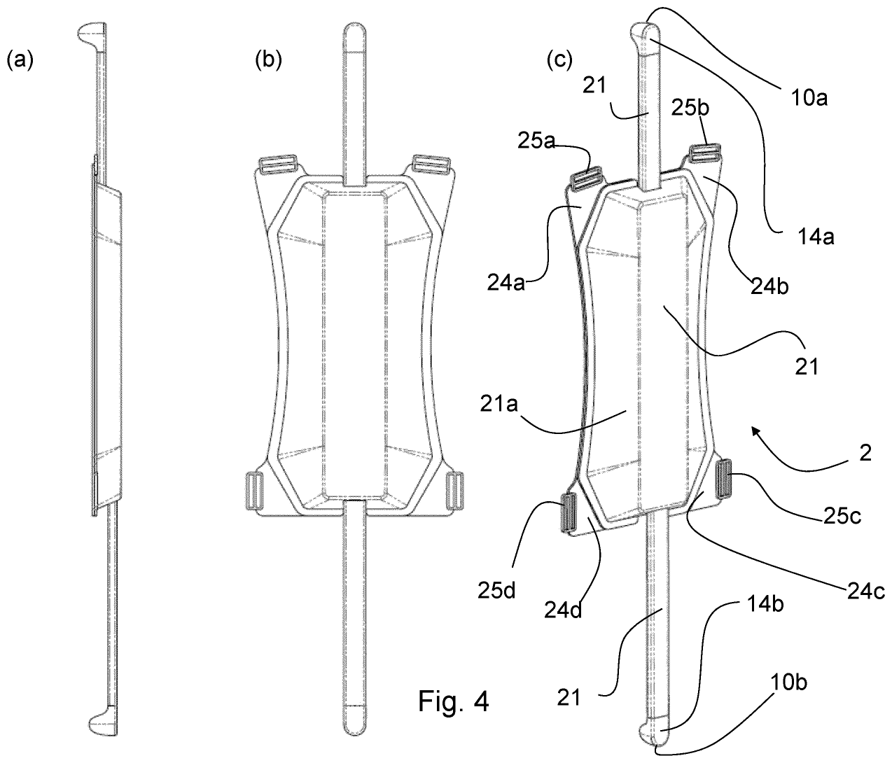

FIG. 4 shows a wearable spinal alignment apparatus 2 incorporating the spinal alignment component 1. FIG. 4(a) is a side elevation view, FIG. 4(b) is a front elevation view and FIG. 4(c) is a perspective view. For clarity, not every feature is labelled where it appears multiple times in FIGS. 4(a)-(c).

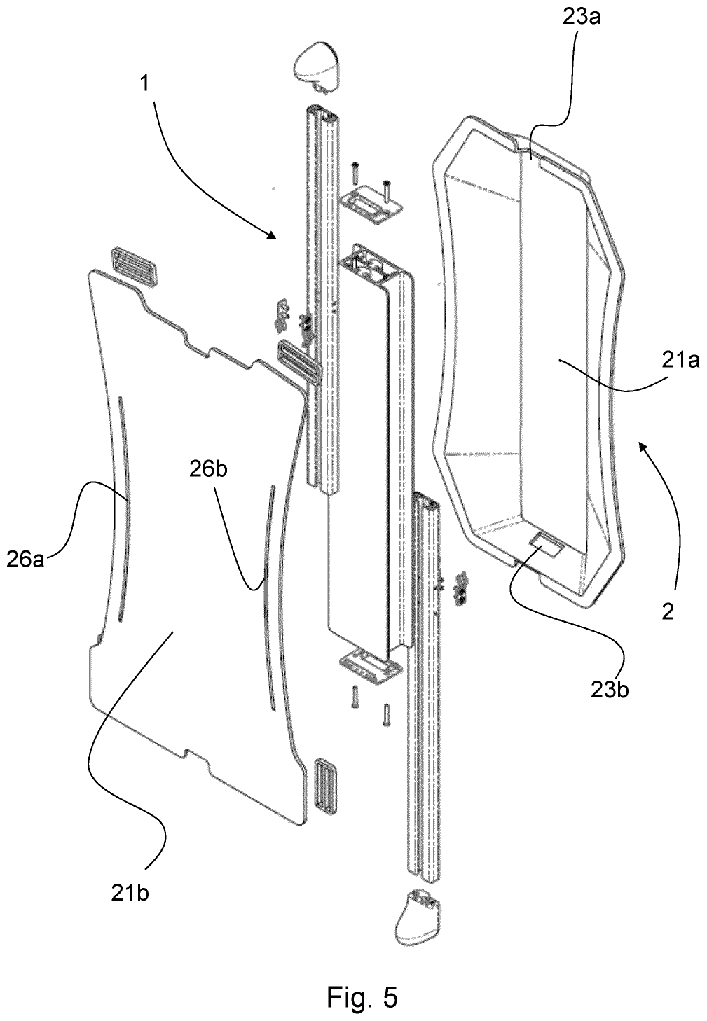

The wearable spinal alignment apparatus 2 is made up of the spinal alignment component 1 within a hollow external housing or shell 21. The hollow shell 21 is made up of a convex front panel 21a which is made of a semi-rigid thermoformed EVA foam and a planar back panel 21b (shown in FIG. 5) which is made of soft, flexible foam covered in a textile layer. The front panel 21a and back panel 21b are fixed together by stitching between the back panel 21b and an external flange 22 of the front panel 21a.

A first aperture 23a (best seen in FIG. 5) is defined between the front panel 21a and the back panel 21b. A second aperture 23b (best seen in FIG. 5) is defined in the front panel 21a. The apertures 23a and 23b are arranged to receive the first and second rigid elongate members 12a and 12b of the spinal alignment component 1 respectively. The housing 11 of the spinal alignment component 1 resides within the hollow shell 21, and the first and second rigid elongate members 12a and 12b extend out of the housing 11 and outwardly through the apertures 23a and 23b respectively in the hollow shell. When assembling the apparatus the housing 11 of the component 1 is first placed within the front panel 21a, the second rigid elongate member 12b is passed through the second aperture 23b and into the second channel 15b, the first rigid elongate member 12a is passed through the first aperture 23a and into the first channel 15a, then the back panel 21b is placed against the front panel 21a and stitched into place along the flange 22 such that the first rigid elongate member 12a passed through the thus-created aperture 23a between the front panel 21a and back panel 21b.

The back panel 21b includes protruding flexible corner portions 24a, 24b, 24c and 24d which carry buckles 25a, 25b, 25c and 25d. The buckles are designed to receive the ends of straps (not shown) which pass around the shoulders and waist of a wearer to secure the wearable spinal alignment apparatus 2 to the wearer's torso, with the back panel 21b held against the wearer's back and the front panel 21a facing outwards away from the wearer.

FIG. 5 is an exploded view of the wearable spinal alignment apparatus 2 showing the component 1 positioned within the hollow shell 21. Aperture 23b in the front panel 21a of the shell 21 can be seen. In addition, elongate apertures or slits 26a and 26b are provided along a peripheral portion of the back panel 21b. The slits 26a and 26b provide access points for the wearer to access the interior of the hollow shell 21, in particular cavities that are created within the hollow shell 21 on either side of the housing 11 of the component 1. These cavities offer the wearer a useful storage facility for storing personal possessions while the apparatus is in use. The slits 26a and 26b in FIG. 5 can be opened by pulling the flexible fabric of the rear panel 21b to enlarge the cross section of the slit opening. The resilient nature of the fabric of the back panel means that the cross section of the slit opening returns to a narrow size when the fabric is released, such that no specific closure means is required, although such closure means, for example a zip or buttons, may be provided for additional security.

The hollow design of the shell 21 creates cavities (not shown) within the shell 21 on either side of the housing 11.

The remaining features of FIG. 5 have been described with reference to earlier Figures.

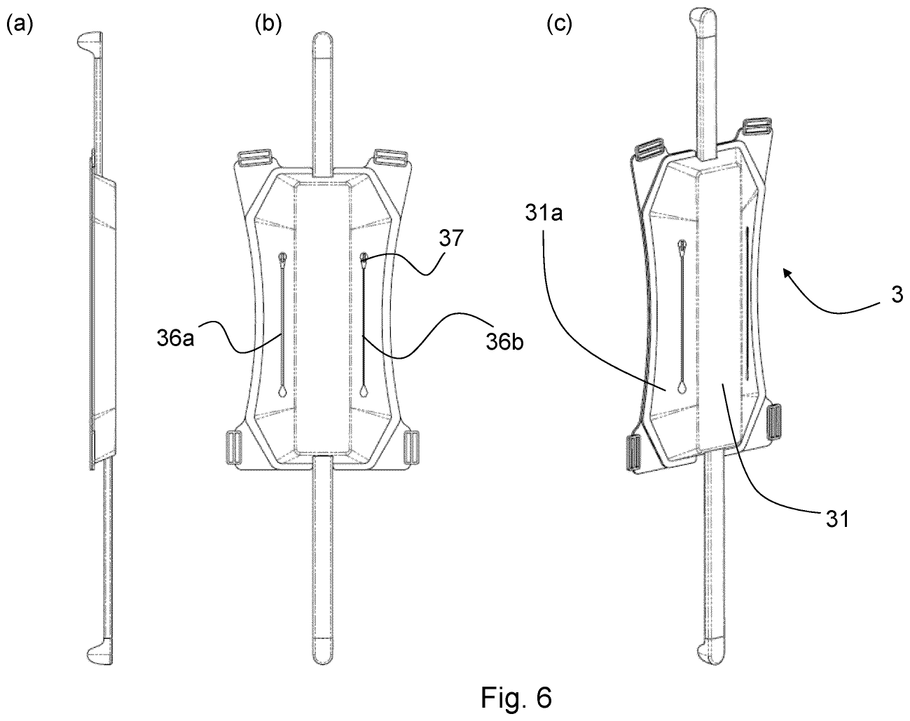

FIG. 6 shows a second embodiment of a wearable spinal alignment apparatus 3. FIG. 4(a) is a side elevation view, FIG. 4(b) is a front elevation view and FIG. 4(c) is a perspective view. All features of the apparatus correspond with those of wearable spinal alignment apparatus 2 shown in FIG. 4, except that wearable spinal alignment apparatus 3, instead of providing elongate apertures in the back panel 31b, provides elongate apertures 36a and 36b in the front panel 31a of the hollow shell 31. Each of the elongate apertures 36a and 36b include a zip mechanism 37 for closure of the aperture.

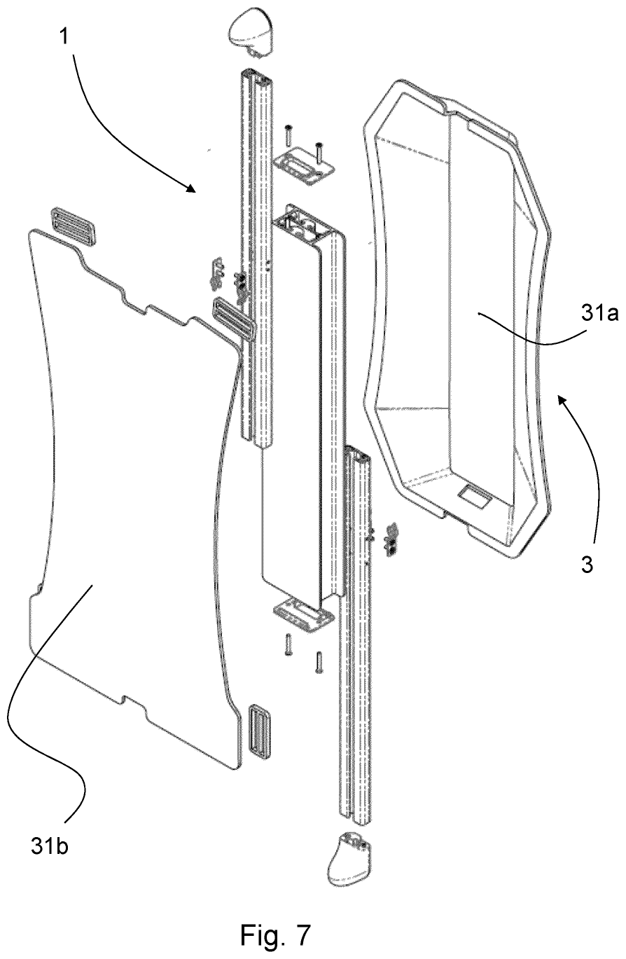

FIG. 7 is an exploded view of the wearable spinal alignment apparatus 3 showing the component 1 positioned within the hollow shell 31.

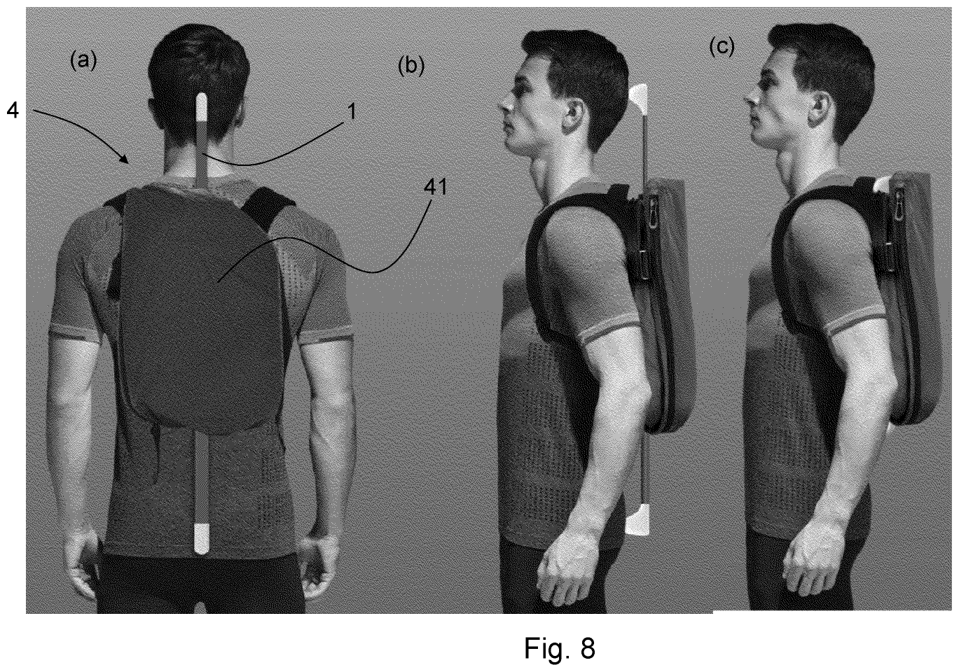

FIG. 8 shows a wearable spinal alignment apparatus 4 comprising the spinal alignment component 1 incorporated into a backpack 41. The component 1 may be incorporated into the backpack 41 by securing the component to the backpack, for example by stitching to irreversibly fix the component in place. FIG. 8(a) shows a rear elevation view with the first and second rigid elongate members 12a, 12b of the component 1 fully extended from the housing 11 such that a first contact portion of the first rigid elongate member 12a lies adjacent the wearer's occipital head and a second contact portion of the second rigid elongate member 12b lies adjacent the wearer's sacral spine. A third contact portion lies on the housing 11 and is secured against the wearer's thoracic spine at all times while the backpack is being worn, by the shoulder straps.

When using the component incorporated into a wearable spinal alignment apparatus, such as the backpack depicted in FIG. 8, the wearer may retract one or both of the first and second rigid elongate members 12a, 12b of the component 1 by pushing them towards the backpack, into the housing 11, during ordinary daily use of the backpack when it is not desirable for the component to be visible to others. FIG. 8(c) shows such a configuration of the apparatus in which both the first and second rigid elongate members 12a, 12b are fully retracted into the housing 11 such that they are minimally visible. This provides the wearer with a versatile piece of apparatus which can be used as an ordinary backpack when desired without the first and second rigid elongate members 12a, 12b being visible to others.

When the wearer wishes to use the apparatus as a posture aid, for example when conducting exercises, they may extend the first and second rigid elongate members 12a, 12b by pulling them away from the housing 11. Alternatively, the wearer may extend only one of the first and second rigid elongate members 12a, 12b while leaving the remaining member in a retracted configuration. For example, the wearer may wish to extend only the first rigid elongate member 12a to contact the occipital head so that they may adopt a seated position and ensure good posture while seated.

One the elongate members are extended, by positioning their body to ensure contact between the occipital head and the first contact portion, and the sacral spine and the second contact portion, the wearer achieves ideal alignment.

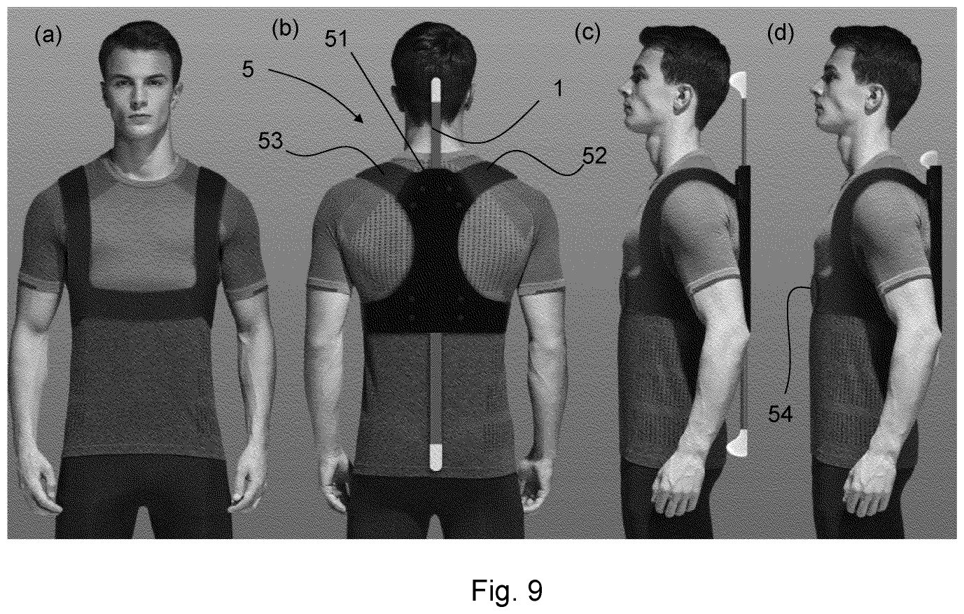

FIG. 9 shows an alternative embodiment of a wearable spinal alignment apparatus 5 comprising the spinal alignment component 1 incorporated into a harness 51 adapted for securement to a user's torso. The harness comprises first and second shoulder straps 52, 53 arranged to encircle the shoulders of a wearer, as shown. The harness also includes a horizontal strap 54 made up of left and right strap components which are attachable across the front of the wearer's torso. In the embodiments shown, the left and right strap components are attachable by hook and loop type fasteners, although any suitable fastening mechanism known to the skilled person may be used. When fastened, the horizontal strap passes around the wearer's torso, for example the wearer's waist to provide additional securement of the component to the wearer's back, minimising the risk of movement of the component away from its contact with the thoracic spine.

In a further embodiment of the invention, the component shown in FIGS. 1-3 includes a plurality of position/orientation sensors. For example, two or more sensors may be provided which measure the position or orientation of the component. In one embodiment, the sensors and contained within the housing 11. For example, there may be a first sensor located at one end of the housing 11, close to (or attached to) the end-cap 19 and a second sensor close to (or attached to) the end-cap (not shown) at the other end of the housing. Such an embodiment facilitates the measurement of position or orientation data regardless of whether the rigid elongate members and extended or retracted.

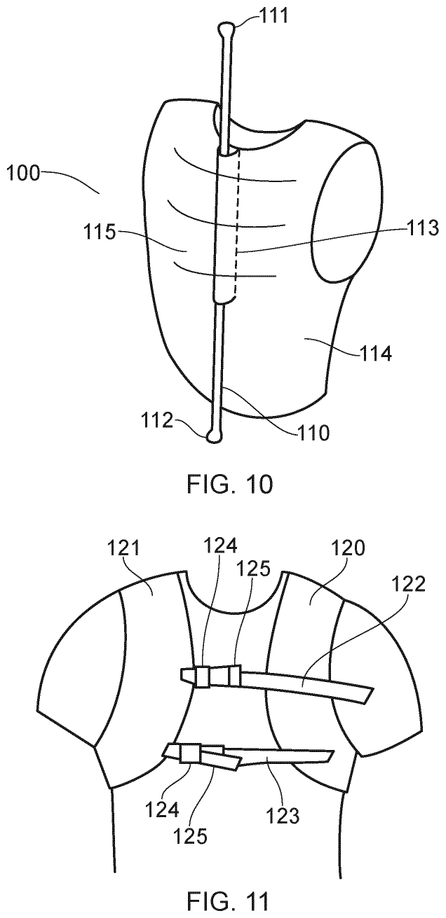

There is shown in FIGS. 10 to 14 a further embodiment 100 of a wearable spinal alignment apparatus 100, which is an embodiment of the apparatus of the seventh aspect of the invention.

FIG. 10 shows the wearable alignment device 100 which comprises a rigid elongate member 110, comprising three contact portions 111, 112, 113. The first 111, second 112, and third 113 contact portions are arranged collinearly along the rigid elongate member.

The rigid elongate member 110 is formed of a rigid plastic, is substantially cylindrical, and is of a length approximately equal to the distance between the occipital head and sacral spine of the wearer. It is attached to the securement system 114 by a fabric sheath 115, which encompasses and retains the intermediate section of the elongate member. In an alternative embodiment, the rigid elongate member 110 is replaced by the spinal alignment component as described above in reference to FIGS. 1-3, with the housing of the component attached to the securement system within the sheath 115.

FIG. 11 shows the securement system 114 comprising left and right shoulder loops 120, 121 and two horizontal securement straps 122, 123. The shoulder loops 120, 121 are disposed to encircle the shoulders of a wearer, and the two horizontal securement straps 122, 123 connect the left and right shoulder loops across the front of the wearer. The horizontal securement straps 122,123 comprise a buckle and clasp fastener 124 to temporarily and reversibly connect the left and right shoulder loops, and a friction based adjuster 125 to reversibly adjust the length of the horizontal securement straps.

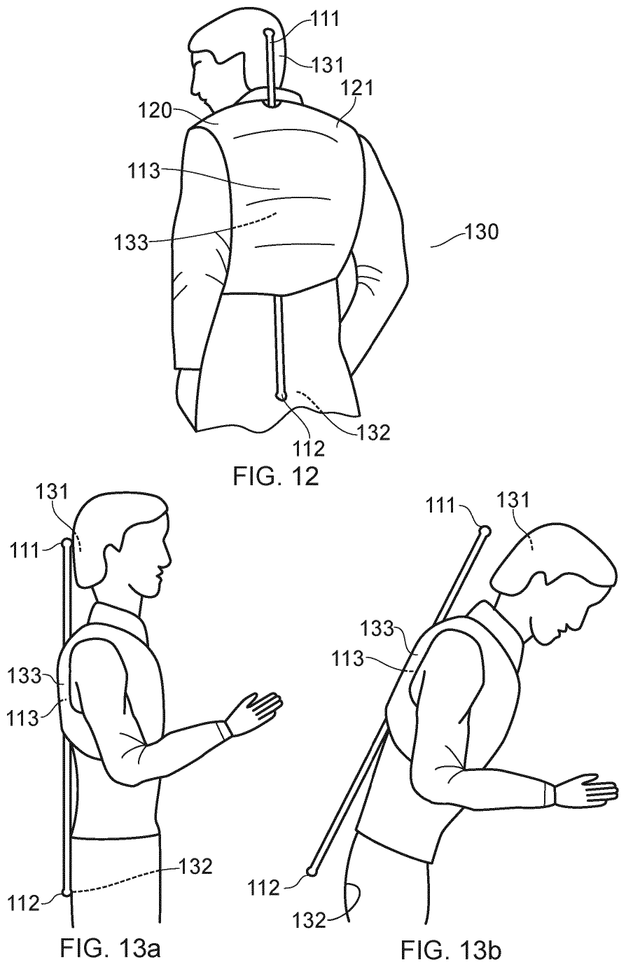

FIGS. 12, 13a and 13b show a wearer 130, wearing the first embodiment of the wearable alignment device 100. The wearer puts the device on by releasing the fasteners 124 to disconnect the horizontal securement straps 122,123 and loosens the adjuster 125 to increase the length of the horizontal securement straps 122, 123. The wearer puts their left arm through the left shoulder loop 120, and puts their right arm through the right shoulder loop 121. The left and right shoulder loops 120, 121 encircle the wearer's shoulders, and the rigid elongate member 110 is positioned about the wearer's back.

The wearer fastens the horizontal securement straps 122, 123 across their front by engaging the buckle and clasp fastener 124. The wearer adjusts the length of the horizontal securement straps by pulling the strap through the friction based adjuster 125, in order to securely locate the rigid elongate member 110 in the centre of their back. The third contact portion 113 is secured against the wearer's thoracic spine 133. The device does not comprise any means to secure the rigid elongate member to the wearer's waist or head.

While the first contact portion 111 is disposed about the occipital head 131, the first contact portion is only in contact with the occipital head when the wearer is in ideal alignment. The wearer can therefore rotate, nod, shake and move their head freely with minimal restriction from the device.

While the third contact portion 113 is disposed about the sacral spine 133, the third contact portion is only in contact with the sacral spine when the wearer is in ideal alignment. The wearer can therefore rotate, bend and move their hips and lower back freely with minimal restriction from the device.

FIG. 13a shows the wearer positioned in ideal alignment. The first, second and third contact portions 111, 112, 113 are in contact with the occipital head, sacral spine and thoracic spine 131, 132, 133. FIG. 13b shows the wearer not positioned in ideal alignment. Only the third contact portion 113 is in contact with the thoracic spine 133, and the first and second contact portions 111, 112 are not in contact with the occipital head and sacral spine 131, 132.

This demonstrates that if the wearer moves away from ideal alignment, at least one of the first 111 or second 112 contact portions will move away from the occipital head 131 and sacral spine 132 respectively. The wearer independently moves their body into ideal alignment, as there is no securing means around the wearer's head or waist to force them into ideal alignment. The wearer uses tactile feedback from the rigid elongate member to determine when ideal alignment is achieved. The wearer undertakes everyday activities and exercise while wearing the device and uses the tactile feedback to maintain ideal alignment. They thereby mentally and physically adapt to maintaining ideal alignment and correct posture while undertaking everyday activities and exercise.

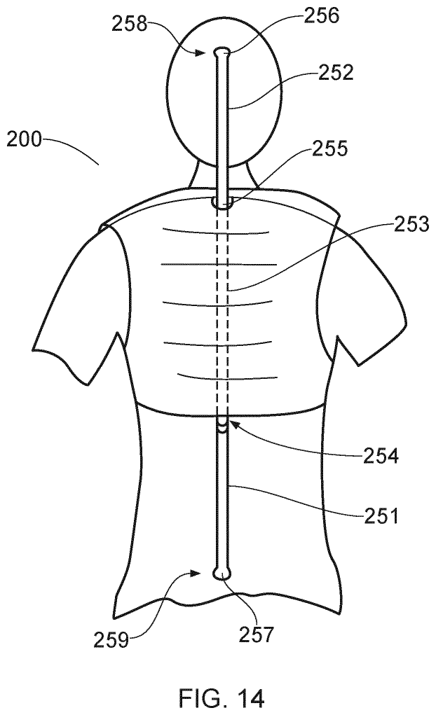

Referring to the drawings, there is shown in FIG. 14 a second embodiment of the wearable alignment device 200.

FIG. 14 shows the wearable alignment device 200, which comprises the features of the first embodiment of the wearable alignment device discussed herein. FIG. 14 shows the rigid elongate member positioned between the shoulder blades and alongside the spine of the wearer.

The device further comprises a lower extendable elongate section 251 and an upper extendable elongate section 252. The extendable elongate sections are concentric with the main body of the rigid elongate member 253 and extend telescopically from therein to adjust the total length of the rigid elongate member. A lower securing clip 254 and upper securing clip 255, fix the extension of the extendable elongate sections 251, 252 from the main body of the rigid elongate member 253. The clips hold the extendable elongate sections rigid to the main body of the rigid elongate member, and maintain the collinearity of the contact portions. The clips are friction lock clips, operable by a flip lock.

The wearer adjusts the extension of the extendable elongate sections 251, 252 by opening the flip lock of the friction lock clips 254, 255 and sliding the extendable elongate sections in or out of the main body of the rigid elongate member 253. The wearer adjusts the total length of the rigid elongate member so that the first contact portion is disposed proximal their occipital head and the second contact portion is disposed proximal their sacral spine, and also so that the peripheries of the rigid elongate member do not protrude below the wearer's waist or above the wearer's head.

FIG. 14 shows a first and second sensors 256, 257 disposed about the first and second contact portions 258, 259. The sensors 256, 257 comprises a force sensor and a wireless communication means, to sense when a wearer's body is in contact with the contact portion and communicate that data to a user device.

The device also includes an inclinometer (not shown) which senses when the rigid elongate member is aligned with the vertical and feeds this back to the user via communication with a user device.

The wearer uses feedback from the user device to determine when ideal alignment is achieved. The wearer uses recorded feedback from the user device to review when ideal alignment is achieved or not achieved during everyday activities and exercise. They thereby mentally and physically adapt to maintaining ideal alignment and correct posture while undertaking everyday activities and exercise.

Although the invention has been explained in relations to two embodiments, it is to be understood that many other possible embodiments can be made without departing from the scope of the present invention. It is, therefore, contemplated that the appended claim or claims will cover such embodiments that fall within the scope of the invention.

### Further Aspects and Embodiments of the Disclosure

The following numbered clauses define further aspects and embodiments of the present disclosure:

1. A wearable alignment device, comprising a rigid elongate member comprising first, second and third contact portions disposed along its length, such that when the device is worn by a wearer the first, second and third contact portions are arranged collinearly and disposed to contact the wearer's occipital head, sacral spine and thoracic spine respectively; and

a securement system comprising left and right shoulder loops adapted to encircle the wearer's left and right shoulders, and at least one horizontal securement strap adapted to connect the left and right shoulder loops across the wearer's front;

wherein when the device is worn by a wearer the securement system is disposed to secure the third contact portion in contact with the thoracic spine, and the first and second contact portions remain movable away from the occipital head and sacral spine respectively.

2. The wearable alignment device according to clause 1, wherein the wearable alignment device does not comprise a securement strap arranged to encircle the wearer's waist.

3. The wearable alignment device according to either clause 1 or clause 2, wherein the wearable alignment device does not comprise a securement strap arranged to encircle the wearer's head.

4. The wearable alignment device according to any one of clauses 1 to 3, wherein at least one horizontal securement strap comprises a fastening means, wherein the fastening means is operable to reversibly detach the horizontal securement strap from across the wearer's front.

5. The wearable alignment device according to any one of clauses 1 to 4, wherein at least one shoulder loops comprises a fastening means, wherein the fastening means is operable to reversibly detach the shoulder loop from encircling the wearer's shoulder.

6. The wearable alignment device according to either clause 4 or clause 5, wherein the fastening means comprises a buckle and clasp.

7. The wearable alignment device according to any one of clauses 1 to 6, wherein at least one horizontal securement strap comprises an adjustment means, wherein the adjustment means is operable to reversibly adjust the length of the horizontal securement straps.

8. The wearable alignment device according to any one of clauses 1 to 7, wherein at least one shoulder loop comprises an adjustment means, wherein the adjustment means is operable to reversibly adjust the length of the shoulder loop.

9. The wearable alignment device according to either clause 7 or clause 8, wherein the adjustment means comprises a friction based adjuster.

10. The wearable alignment device according to any one of clauses 1 to 9, wherein the rigid elongate member is cylindrical.

11. The wearable alignment device according to any one of clauses 1 to 10, wherein the rigid elongate member is comprised of a suitable metal, such as aluminium.

12. The wearable alignment device according to any one of clauses 1 to 11, wherein the rigid elongate member comprises at least one extendable elongate section, adapted to reversibly adjust the length of the rigid elongate member and the extendable elongate section is secured rigidly and collinear to the main body of the elongate member.

13. The wearable alignment device according to clause 12, wherein the rigid elongate member comprises two extendable elongate sections.

14. The wearable alignment device according to any one of clauses 1 to 13, wherein the securement system is incorporated into a bag.

15. The wearable alignment device according to any one of clauses 1 to 13, wherein the securement system is incorporated into clothing.

16. The wearable alignment device according any one of clauses 1 to 15, further comprising a sensing means, wherein the sensing means is adapted to sense contact between a contact portion and the wearer's body, and to generate sensor data, and a communication means, wherein the communication means is adapted to communicate the sensor data with one or more user devices.

17. The wearable alignment device according to clause 16, wherein the sensing means is disposed about the first and second contact portions.

18. The wearable alignment device according to clause 16, wherein the sensing means is disposed about the first contact portion.

19. A method for using a wearable alignment device to improve the alignment of a wearer according to any one of clauses 1 to 18.

20. A method of manufacturing a wearable alignment device according to any one of clauses 1 to 19.

1. A spinal alignment component adapted for incorporation into a wearable spinal alignment apparatus, the spinal alignment component comprising:
a first rigid elongate member comprising a first distal end; and
a second rigid elongate member comprising a second distal end;
wherein the first rigid elongate member and the second rigid elongate member are independently movable relative to one another to adjust the displacement between the first distal end and the second distal end.

2. A spinal alignment component according to claim 1, further comprising a housing which receives each of the first rigid elongate member and second rigid elongate member, wherein each of the first rigid elongate member and second rigid elongate member are reversibly extendable from the housing, and wherein each of the first distal end and the second distal end remain external to the housing when the first rigid elongate member and second rigid elongate member are retracted into the housing.

3. A spinal alignment component according to claim 2, wherein each of the respective first and second rigid elongate members are slidably received within the housing, each of the respective first and second rigid elongate members being axially slidable within the housing.

4. A spinal alignment component according to claim 2, wherein the first rigid elongate member is extendable from the housing in a first direction and the second rigid elongate member is extendable from the housing in a second direction, wherein the first direction is diametrically opposed to the second direction.

5. A spinal alignment component according to claim 2, wherein the housing is a rigid elongate housing defining a first channel and a second channel, the first channel being adapted to receive the first rigid elongate member and the second channel being adapted to receive the second rigid elongate member, wherein the respective first and second rigid elongate members are each slidably received within the housing.

6. A spinal alignment component according to claim 5, wherein the first channel and the second channel are parallel channels defined by the housing and separated by a partition wall.

7. A spinal alignment component according to claim 5, wherein each of the first channel and the second channel extend along the entire axial length of the housing.

8. A spinal alignment component according to claim 2, comprising a friction fit mechanism between the housing and each of the first rigid elongate member and second rigid elongate member.

9. A spinal alignment component according to claim 8, wherein the friction fit mechanism comprises a resilient protrusion on an outer surface of each of the first and second rigid elongate members which cooperates with a linear array of teeth distributed along an inner surface of the housing, such that the protrusion moves along the linear array of teeth as the rigid elongate member is moved relative to the housing, and interference between the resilient protrusion and one or more teeth from the linear array facilitates the temporary securement of the rigid elongate member in a specific position relative to the housing.

10. A spinal alignment component according to claim 1, wherein the component is adapted for incorporation into a wearable spinal alignment apparatus by comprising attachment means for securement to a wearable component.

11. A spinal alignment component according to claim 2, wherein the component is adapted for incorporation into a wearable spinal alignment apparatus by comprising attachment means on the housing for securement to a wearable component.

12. A spinal alignment component according to claim 11, wherein the attachment means comprise one or more flanges extending outwardly from a main body of the housing, the one or more flanges each optionally comprising one or more apertures through which a securement means may be passed.

13. A spinal alignment component according to claim 1, wherein the displacement between the first distal end and the second distal end is adjustable to correspond with the distance between the occipital head and the sacral spine of a user when the user is in ideal alignment.

14. A spinal alignment component according to claim 1, wherein each of the first distal end and the second distal end comprise a sensing means, wherein the sensing means is adapted to sense contact between a contact portion and the wearer's body, and to generate sensor data, and a communication means, wherein the communication means is adapted to communicate the sensor data with one or more user devices.

15. A spinal alignment component according to claim 1, wherein each of the first and second elongate components comprises a terminal region having an enlarged profile adjacent to the respective first distal end and second distal end.

16. A spinal alignment component according to claim 15, wherein the enlarged profile is provided by a protrusion of the terminal region extending laterally in one direction.

17. A wearable spinal alignment apparatus comprising the spinal alignment component according to claim 1, and a securement system to attach the apparatus to the torso of a wearer.

18. A wearable spinal alignment apparatus according to claim 17, comprising an external housing, wherein the spinal alignment component is held within the external housing, the external housing being adapted for securement to the torso of a wearer by a securement system comprising one or more straps, and the external housing defining first and second apertures through which each of the first and second rigid elongate members respectively pass.

19. A wearable spinal alignment apparatus according to claim 18, wherein one or more cavities are defined between the spinal alignment component and an inner wall of the external housing.

20. A wearable spinal alignment apparatus according to claim 18 or 19, wherein the spinal alignment component is fixedly attached to the external housing.

21. A wearable spinal alignment apparatus according to claim 20, wherein the spinal alignment component is fixedly attached to the external housing by stitching.

22. A wearable spinal alignment apparatus comprising the spinal alignment component according to claim 1, and a backpack, wherein the spinal alignment component is fixedly attached to the backpack.

23. A wearable spinal alignment apparatus according to claim 22, wherein the spinal alignment component is fixedly attached to the backpack by stitching.

24. A wearable spinal alignment apparatus comprising the spinal alignment component according to claim 1, and a harness adapted to secure the apparatus to the torso of a user.

25. Use of the spinal alignment component according to claim 1 to correct the posture of a user.

26. Use of the spinal alignment component according to claim 1 in a wearable alignment device or apparatus.

27. Use according to claim 26, wherein the wearable alignment device or apparatus comprises a backpack.

28. A spinal alignment component adapted for incorporation into a wearable spinal alignment apparatus, wherein the spinal alignment component comprises sensing means to detect one or more of movement, position and orientation of a wearer during use of the component.

29. A wearable spinal alignment apparatus comprising the spinal alignment component according to claim 28, and a securement system to attach the apparatus to the torso of a wearer.

30. A wearable spinal alignment apparatus according to claim 29, wherein the apparatus is a backpack.

31. Use of the wearable spinal alignment apparatus according to claim 17 to correct the posture of a user.
